(19) United States
(12) Patent Application Publication
(10) Pub. No.: US 2010/0215660 A1
(43) Pub. Date: Aug. 26, 2010

(54) KRUPPEL-LIKE FACTORS AND FAT REGULATION

(76) Inventors: Sarwar Hashmi, Holmdel, NJ (US); Chuan Yang, Jackson Heights, NY (US); Jun Zhang, Oakland Gardens, NY (US); Cheng-Han Huang, Flushing, NY (US)

Correspondence Address: K&L Gates LLP, 1900 MAIN STREET, SUITE 600, IRVINE, CA, 92614-7319, US

(21) Appl. No.: 12/710,910

(22) Filed: Feb. 23, 2010

Related U.S. Application Data
(60) Provisional application No. 61/154,748, filed on Feb. 23, 2009.

(51) Int. Cl. A61K 39/395; A61K 31/7088; A61K 38/16; A61K 38/00; C12N 5/10

(52) U.S. Cl. 424/141.1; 514/44 R; 514/44 A; 514/12; 424/130.1; 514/2; 435/325

(57) ABSTRACT

Disclosed herein are methods and cell lines used in fat regulation. The methods and cell lines incorporate Krüppel-like factors including, without limitation, klf-1 and klf-3.

### CROSS REFERENCE TO RELATED APPLICATIONS

The present application claims the benefit under 35 USC 119(e) to U.S. Provisional Patent Application No. 61/154,748 filed Feb. 23, 2009, the entire contents of which are incorporated by reference herein.

### FIELD OF THE INVENTION

Disclosed herein are methods and cell lines used in fat regulation. The methods and cell lines incorporate Krüppel-like factors including, without limitation, klf-1 and klf-3.

### BACKGROUND OF THE INVENTION

Energy stored in the form of fat is a basic property universal to animals from Caenorhabditis elegans (C. elegans) to humans, allowing organisms to continue life during periods of fasting or starvation. A complex multi-factorial trait driven by natural selection and food availability, fat storage is highly regulated by, and dynamically balanced with, energy consumption in physiological settings; its perturbation in either excess (obese) or deficit (lipodystrophy) has devastating pathologic consequences in the homeostasis and fitness of an organism. In humans, the obese state preconditions insulin resistance and impairs pancreatic islet β-cell function, two hallmarks of type 2 diabetes, a chief metabolic disease and severe threat to the health of worldwide populations. Obesity also may result in reproductive deficiency and cardiovascular disease. Hence, understanding the cellular origins and regulatory mechanisms of fat storage in model organisms, such as C. elegans, should help unravel the molecular targets underlying its signal transduction, gene expression, and pathway coordination, yielding new approaches to therapeutic applications.

C. elegans stores fat mainly in cells of its intestine, a derivative tissue of the developing layer of endoderm. Prior genome-wide RNA interference (RNAi) studies have uncovered a plethora of genes affecting lipid metabolism, which underscores the conserved nature of molecular mechanisms in fat storage in worms and mammalians. It was found that the suppression of 305 genes reduced body fat, while the suppression of 112 genes either enhanced fat storage or enlarged fat droplet size. The products of these genes are metabolic enzymes, transcription factors, signaling modules, and nutrient transporters, reflecting a wide range of biochemical identities and pathway activities. However, the mechanisms by which these factors act either positively or negatively in the modulation of fat storage remain largely unexplored. Direct inactivation of those C. elegans genes homologous to known mammalian lipid metabolism regulatory factors have demonstrated the existence of molecular players that serve as master switches at the level of gene transcription and/or signal transduction. Examples include the transcription factors SREBP and C/EBP, which cause a lipid-depleted phenotype when mutated. The C. elegans Δ9-desaturase fat-5, fat-6, and fat-7 genes are expressed in the intestine where they undergo strict regulation by a transcription factor, NHR-80, and maintain an optimum fatty acid composition in C. elegans. In contrast to such positive regulators, little is known about the negative regulators of fat storage in regards to their mode of action and mechanisms of regulation. Several reports from mouse and cell culture studies have suggested that the differentiation of preadipocytes into adipocytes is regulated by a complex network of transcription factors which synchronize the expression of many proteins. These proteins are responsible for determining the shape of a mature fat cell. Members of the Krüppel-like factor (KLF) family form a subset of a broad class of proteins containing C₂H₂ zinc fingers, the most abundant motif in transcription factors. Although vertebrate KLF is involved in many physiological roles, few mammalian KLFs have been identified as key molecules in controlling adipocyte differentiation or adipogenesis though high levels of RNA or KLF proteins are found in adipocytes.

Mammalian KLFs encode a conserved set of transcription factors that are expressed in many cell types. These transcription factors perform diverse roles in cell proliferation, differentiation, and development. Currently, the human KLF family consists of 17 members that are related to the SP1-like family of transcription factors. The members of the KLF family share a high conservation within their C-terminal C₂H₂ zinc finger binding domains, whereas their N-termini contain domains for transcriptional activation or repression as well as protein-protein interaction. The KLF bind to specific CACCC/GC/GT-boxes that are found in the regulatory regions of genes and thus function in the regulation of various biological processes including cell proliferation, apoptosis, cell differentiation, and early embryonic development.

Determining the function and regulation of KLFs will yield important clues about their basic mechanism in the conserved pathways of embryonic and postembryonic development. Understanding these mechanisms can lead to rational function-based approaches for drug design toward KLF-associated disease.

### SUMMARY OF THE INVENTION

Disclosed herein are compositions and methods of regulating fat accumulation comprising upregulating or downregulating the activity of at least one Krüppel-like factor.

In one embodiment, a method is provided of regulating fat accumulation comprising upregulating or downregulating the activity of at least one Krüppel-like factor (KLFs).

In another embodiment, a method is provided of suppressing or stimulating cell differentiation processes comprising upregulating or downregulating the activity of at least one KLF.

In another embodiment, a cell line transfected with at least one KLF gene is provided.

In another embodiment, the upregulating or downregulating the activity of at least one KLF comprises administering an agent that potentiates or inhibits the expression of at least one KLF gene. In another embodiment, the agent is selected from the group consisting of DNA, RNA, cDNA, siRNA, and shRNA.

In another embodiment, the upregulating or downregulating the activity of at least one KLF comprises administering an agent that potentiates or inhibits the activity of at least one KLF proteins. In another embodiment, the agent is selected from the group consisting of proteins, monoclonal antibodies, polyclonal antibodies, peptides, and small molecules.

In another embodiment, the KLF is a human KLF selected from the group consisting of klf-1, klf-2, klf-3, klf-4, klf-5, klf-6, klf-7, klf-8, klf-9, klf-10, klf-11, klf-12, klf-13, klf-14, klf-15, klf-16 and klf-17.

In another embodiment, the KLF is a Caenorhabditis elegans KLF selected from the group consisting of klf-1, klf-2, and klf-3.

In another embodiment, the upregulating comprises stimulating at least one KLF gene activity.

In another embodiment, the downregulating comprises mutating at least one gene encoding KLF proteins or genes that are expressed by activity of the KLFa.

In another embodiment, the downregulating comprises administering an agent that blocks a biological site required for the activity of the KLFa or otherwise inhibits the activity of the KLFa.

In another embodiment, the agent is a klf-1 antagonist, a klf-3 antagonist, DNA, RNA, cDNA, protein, a monoclonal antibody, a polyclonal antibody or a peptide.

### BRIEF DESCRIPTION OF THE FIGURES

FIG. 1A depicts amino acid sequence alignments of the C-terminal zinc finger domains of C. elegans Krüppel-like factor (KLF)-related proteins klf-1 (SEQ ID NO:1), mua-1a (SEQ ID NO:109), mua-1b (SEQ ID NO:110) and F53F8.1 (KLF-3, SEQ ID NO:3). Amino acid identity is marked with black. Asterisks mark the invariant zinc-chelating residues. Three zinc fingers are marked. The upside-down black triangles indicate those residues that contact the DNA. The Ce stands for C. elegans.

FIG. 1B depicts genomic organization of the C. elegans Ce-klf-1 gene.

FIG. 1C depicts a translational fusion construct created by fusion of a 2-kb promoter region upstream of the klf-1 ATG and its full coding sequence consisting of eight exons in frame with gfp reporter (pHZ109). Exons are indicated as shaded boxes in black, the gray boxes indicate 5′ and 3′ UTR, and the numbers under the boxes indicate their sizes in base pairs (bp). Promoters and the introns between the exons are indicated by solid line. The numbers above the solid line indicate sizes in base pairs (bp).

FIG. 2 depicts a temporal pattern of klf-1 gene expression as determined by real-time PCR. Note that Ce-klf-1 transcripts are low in embryos but increased several folds in the larval stages and decreased again in adult.

FIG. 3 depicts the spatiotemporal expression of Ce-klf-1::gfp. The images are merged images of differential interference contrast microscopy (DIC) and green fluorescent protein (gfp) for clarity. The gfp fluorescence signal is observed in (I) anterior region of the intestine of young larvae and (II) the intestine of the posterior region of the young larvae; (III) Egg-laying adult showing gfp expression in intestine (solid line) and a few hypodermal cells (arrows); (IV) head region of older adult showing gfp expression in intestine (solid line) and a few neurons (arrows). All images are anterior to the top and ventral to the left.

FIG. 4A depicts phenotypes of C. elegans klf-1 RNAi worms. (I) Control gonad showing normal spermatheca (arrow), normal oocytes (solid line), and germline (solid line); (II) egg-laying hermaphrodites showing many dead cells in the uterus (arrows); (III) klf RNAi adult hermaphrodite stained with acridine orange (AO) shows increased number of germline apoptosis shown in white (arrowhead and solid line); (IV) DIC image of same animal showing many dead cells (solid line); (V) AO staining was barely seen in wild-type worm.

FIG. 4B depicts Ce-klf-1 RNAi and wild-type animals showing fat staining. (I) Low accumulation of fat in wild-type animal; (II) extensive fat accumulation along the intestine (solid line; arrowheads indicating individual fat body) in Ce-klf-1 (RNAi) L4 larva; (III) much more extensive accumulation of fat along the intestine (solid line; arrowheads showing individual fat body) in older adult Ce-klf-1 (RNAi) adult worm. All images are anterior to the top and ventral to the left.

FIG. 5 depicts amino acid sequence alignments of the C-terminal zinc finger domains of C. elegans KLFs proteins (Ce-klf-3 (SEQ ID NO:3), Ce-klf-2 (SEQ ID NO:2), and Ce-klf-1 (SEQ ID NO:1)) with human KLF proteins (HsKLF1 (SEQ ID NO:4) and HsKLF7 (SEQ ID NO:12) (FIG. 5A). Amino acid identity is marked with black. Asterisks denote the invariant zinc-chelating residues in the three zinc fingers and black diamonds indicates those DNA-contacting residues. FIGS. 5B and 5C depict the genomic organization of C. elegans klf-3a and klf-3b genes. Black boxes indicate exons and grey boxes 5′ and 3′UTR. The exon size in base-pair (bp) is numbered under the box. Promoters and introns are indicated by a solid line above which the size of introns is numerated. FIG. 5D is a diagram of the klf-3::gfp fusion construct, pHZ122. The construct contains the 1.0-kb upstream promoter and full-length coding sequence of klf-3 fused in frame with the gfp reporter.

FIG. 6. depicts temporal expression pattern of the klf-3 gene as determined by qRT-PCR. The levels of klf-3 mRNA in each developmental stage were measured, using the ama-1 gene as an internal control. Total RNA samples used for cDNA synthesis were isolated from mixed-stage embryos, synchronized larvae, and adult populations, respectively. Note that klf-3 transcript is low in embryos but increased steadily in the larval stages and decreased again in adult. Each experimental point was repeated at least twice.

FIG. 7 depicts images of klf-3::gfp expression during development in transgenic lines of C. elegans carrying the pHZ122 construct for the klf-3::gfp fusion gene. Klf-3::gfp expression is seen in (I) un-hatched larva, which is still inside the eggshell (solid line); (II) intestinal cells in young adult hermaphrodite (arrows); (III) intestinal segments covering the mid body and tail region of young adult worm (solid line), but in gonads (arrows) and vulva (v); (IV) intestine of egg-laying hermaphrodite (solid line); and (V) intestine of a male worm (solid line). Transgenic worms were observed and photographed using Axioskop 2 plus fluorescent microscope with appropriate filter sets (400× magnifications). Expression of GFP is merged with DIC images for clarity.

FIG. 8 depicts the characterization of klf-3 mutant worms. FIG. 8A is a diagram of genomic deletion identified in ok1975 and rh160 mutant alleles. The deletion is denoted with a shaded bar and its size is shown in bp. FIG. 8B depicts klf-3 (ok1975) worms with the following distinctive phenotypes: (I) WT gonad has normal spermatheca (arrowhead), oocytes (small arrows), embryos (solid line), and germ cells (arrows); (II) on the 3rd day of adulthood, the semi-sterile mutant hermaphrodites show egg-laying defects with uterus containing many degenerated embryos (arrows); (III) in sterile worms, DAPI staining (in white) reveals the absence of normal morphology in the germline and oocyte area of the gonad, and the disorganized clump of cells is found scattered in the gonad (arrows) and around vulva opening (v); (IV) the oocyte region of the gonad arm of the sterile worm is filled up with small morphologically abnormal oocytes (arrows), and is associated with gonad degeneration; (V) some older egg-laying worms show muscle detachment near vulva (v) opening (arrows); All photographs were taken using Nomarski optics (400× magnifications).

FIG. 9A depicts fat storage and the morphological appearance of klf-3 (ok1975) mutant worms. (I) Low fat content in WT L4; (II) extensive fat accumulation in klf-3 (ok1975) larvae; and (III) enhanced Sudan black staining in klf-3 (ok1975) adult hermaphrodite. All photographs were taken using Nomarski optics (400× magnifications). FIG. 9B depicts electron micrographs of thin sections of mutant and WT worms. (I) Few small lipid droplets (star) are present in the WT worm; (II) the mutant worm bearing large lipid droplets (star); and (III) another section of mutant worm showing large lipid droplets. Hyp, denotes hypodermis and Int, intestine. The horizontal scale bar is 250 nm.

FIG. 10 depicts the total lipid content, comprising triglycerides (FIG. 10A), total cholesterol (FIG. 10B), phospholipids (FIG. 10C) and cholesterol esters (FIG. 10D) in both klf-3 (ok1975) mutants and wild-type worms. Error bars indicate standard deviations.

FIG. 11 depicts the fatty acid composition in the klf-3 (ok1975) mutant (FIG. 11A) as compared to C. elegans (N2) wild-type strain (FIG. 11B). Gas chromatography (GC) profiles: retention time at X-axis; intensity of signal is shown at Y-axis. The arrow points to the peaks corresponding to stearic acid (C, 18.0) and linoleic acid (C18:2w6c) in both wild type and klf-3 (ok1975) mutant. Note that these peaks are much lower in klf-3 mutant than wild type worm. Arrowhead indicates a slightly lower peak of palmitic acid (C, 16.0) in klf-3 mutant. GC analysis were performed on 5 samples of klf-3 (ok1975) mutant and adult population of wild type (N2) strain collected on 5 different days.

FIG. 12 depicts the deregulation of genes for lipid metabolism in the klf-3 (ok1975) mutant. The level of expression of multiple genes (designated at bottom) on the klf-3 mutant background was measured by real-time PCR. Lines at the top of each bar represent standard error of the measurement. Abundance of individual gene is expressed as relative to WT at scale “1”. The bars above “1” represent up-regulated, while the bars below “1” represent down-regulated genes.

FIG. 13 is a schematic presentation of the fatty acid (FA) desaturation in C. elegans. The genes involved in FA desaturases steps are taken from Van Gilst et al., Nuclear hormone receptor NHR-49 controls fat consumption and fatty acid composition in C. elegans. PLoS Biol. 3 (2005) 301-212 which is incorporated by reference herein for information relating to FA desaturation. Certain genes are up-regulated (fat-2, fat-6, fat-1 and fat-3) while others are down-regulated (pod-2, fasn-1, elo-2 and fat-7) in klf-3 (ok1975) mutant as detected by qRT-PCR. The expression of fat-2 remained unchanged. Fatty acids altered in klf-3 (ok1975) mutant and easily detectable by GC include C18:0, 18:2w6c and C20:2w6c.

FIG. 14A depicts food intake by wild-type (WT, I), eat-2 (II), and klf-3 (III) mutant animals. FIG. 14B depicts the relative fluorescence intensity of the three strains.

FIG. 15 depicts deregulation of genes involved in FA desaturation and transport in the klf-3 (ok1975) mutant. The level of expression of multiple genes (designated at bottom) was measured by real-time PCR. Abundance of individual gene is expressed as relative to WT at scale “1”. The bars above “1” represent up-regulated, while the bars below “1” represent down-regulated genes.

### DEFINITION OF TERMS

The following definition of terms is provided as a helpful reference for the reader. The terms used herein have specific meanings as they related to the present disclosure. Every effort has been made to use terms according to their ordinary and common meaning. However, where a discrepancy exists between the common ordinary meaning and the following definitions, these definitions supercede common usage.

Antibody: As used herein, the term “antibody” includes intact antibodies and any antigen binding fragment (i.e., “antigen-binding portion”) or single chain thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. Each light chain is comprised of a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The terms “monoclonal antibody” or “monoclonal antibody composition” as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

The term “antigen-binding portion” of an antibody (or simply “antibody portion”), as used herein, refers to one or more fragments of an antibody that retain the ability to bind to an antigen. It has been shown that the antigen-binding function of an antibody can be performed by fragments of an intact antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR), e.g., V_{H} CDR3. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Furthermore, the antigen-binding fragments include binding-domain immunoglobulin fusion proteins comprising (i) a binding domain polypeptide (such as a heavy chain variable region, a light chain variable region, or a heavy chain variable region fused to a light chain variable region via a linker peptide) that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The hinge region is preferably modified by replacing one or more cysteine residues with serine residues so as to prevent dimerization. Such binding-domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies.

Biological molecule: As used herein, the term “biological molecule” refers to, but is not limited to, lipids, polymers of monosaccharides, amino acids and nucleotides having a molecular weight greater than 450.

Nucleic acid: The terms “nucleic acid” or “nucleic acid molecules” refer to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof and complementary sequences, as well as the sequence explicitly indicated. The nucleic acid molecules can include any type of nucleic acid molecule capable of mediating RNA interference, such as, without limitation, short interfering nucleic acid (siNA), short hairpin nucleic acid (shNA), short interfering RNA (siRNA), short hairpin RNA (shRNA), micro-RNA (miRNA), and double-stranded RNA (dsRNA). The nucleic acid molecules also include similar DNA sequences. Further, the nucleic acid and nucleic acid molecules of the present invention can contain unmodified or modified nucleotides. Modified nucleotides refer to nucleotides which contain a modification in the chemical structure of a nucleotide base, sugar and/or phosphate. Such modifications can be made to improve the stability and/or efficacy of nucleic acid molecules and are described in patents and publications such as U.S. Pat. No. 6,617,438, U.S. Pat. No. 5,334,711; U.S. Pat. No. 5,716,824; U.S. Pat. No. 5,627,053; U.S. Patent Application No. 60/082,404, International Patent Cooperation Treaty Publication Number (“PCTPN”) WO 98/13526; PCTPN WO 92/07065; PCTPN WO 03/070897; PCTPN WO 97/26270; PCTPN WO 93/15187; Beigelman et al., J. Biol. Chem., 270:25702, 1995; Usman and Cedergren, TIBS. 17,:34, 1992; Usman et al., Nucleic Acids Symp. Ser. 31:163, 1994; Burgin et al., Biochemistry, 3:14090, 1996; Perrault et al. Nature, 344:565-568, 1990; Pieken et al. Science, 253:314-317, 1991; Usman and Cedergren, Trends in Biochem. Sci., 17:334-339, 1992; Karpeisky et al., Tetrahedron Lett., 39:1131, 1998; Earnshaw and Gait, Biopolymers (Nucleic Acid Sciences), 48:39-55, 1998; Verma and Eckstein, Annu. Rev. Biochem., 67:99-134, 1998; Burlina et al., Bioorg. Med. Chem., 5:1999-2010, 1997; Limbach et al., Nucleic Acids Res. 22:2183, 1994; and Burgin et al., Biochemistry, 35:14090, 1996. Such patents and publications describe general methods and strategies to modify nucleic acid molecules and are incorporated by reference herein.

Protein: The terms “polypeptide,” “peptide,” and “protein” are used interchangeably herein to refer to a polymer of amino acid residues. The terms apply to amino acid polymers in which one or more amino acid residues is an artificial chemical mimetic of a corresponding naturally occurring amino acid, as well as to naturally occurring amino acid polymers and non-naturally occurring amino acid polymers. In general, however, the term “peptides” refers to amino acid polymers having less than 25 amino acids; “polypeptides” refers to amino acid polymers from 25 to 100 amino acids in length; and “proteins” refers to amino acid polymers having more than 100 amino acids.

Small molecule: As used herein, the term “small molecule” refers to a molecule that is not a biological molecule. Accordingly, small molecules include, but are not limited to, organic compounds, organometallic compounds, salts of organic and organometallic compounds, saccharides, amino acids and nucleotides. Small molecules further include molecules that would otherwise be considered biological molecules, except that the molecular weight is not greater than 450. Thus, small molecules may be lipids, oligosaccharides, oligopeptides, and olionucleotides, and their derivatives, having a molecular weight of 450 or less. It is emphasized that small molecules can have any molecular weight. They are merely referred to as small molecules because they typically have molecular weights less than 450. Small molecules include compounds found in nature as well as synthetic compounds.

### DETAILED DESCRIPTION

The Caenorhabditis elegans genome predicts three Krüppel-like transcription factors (KLF), which include muscle attachment abnormal proteins, mua-1a (F54H5.4a) and mua-lb (F54H5.4b), gate F53118.1 identified as a homolog of human WTI and a novel gene F56F11.3, which was named Krüppel-like factor 1 (klf-1) (Wormbase; http://www.wormbase.org/). These C. elegans genes encode C₂H₂ zinc finger proteins of the KLF family. In embodiments disclosed herein, C. elegans was used to analyze the function of Ce-klf-1 and Ce-klf-3. C. elegans is an excellent model system for studying functions pertinent to developmental biology because it is complex enough to exhibit many biological properties common to higher multicellular organisms, yet simple enough to be studied in great detail.

Suppression of Ce-klf-1 function by RNA interference (RNAi) resulted in an increase of fat in the intestine of the RNAi worm. This gene disruption also leads to accumulation of dead cells in the germline. The increased fat storage in conjunction with the appearance of Ce-klf-1 expression in the worm's intestine during larval development along with its continued presence in the adult worm suggests a definitive role for C. elegans klf-1 in fat regulation. Therefore, disturbance in this process may lead to increased cell death and thus a defect in phagocytosis of dead cells. The described data reveals important roles for a C. elegans KLF in organismal development.

The described embodiments also provide new genetic insight into fat storage by identifying the C. elegans Krüppel-like factor 3, Ce-klf-3 (mua-1 or F54H5.4) as a hitherto unrecognized key regulator of fat metabolism in C. elegans. This was prompted by the finding that klf-1, a member of the KLF class in the worm, is involved in fat metabolism and in cell death and phagocytosis. Embodiments disclosed herein show that klf-3 is notably distributed in the intestine conforming to its spatiotemporal expression during development and implying its role in intestinal fat metabolism. Embodiments disclosed herein demonstrate through detailed genetic and phenotypic analyses that the two alleles of klf-3 mutant, klf-3 (ok1975) and klf-3 (rh160), carry different genomic deletions, with each exhibiting distinctive loss-of-function phenotypes. A deletion in klf-3 (rh160) II mutants caused the majority of the animals to grow poorly and fail to reach adulthood. A molecular analysis of the klf-3 (rh160) allele confirmed that the extensive genetic disruption that occurred in this mutant worm affects few neighboring genes in addition to klf-3. Significantly and unexpectedly, the klf-3 (ok1975) allele, characterized by a 1658-bp deletion in the klf-3 gene that spans the 3′ end of exon 2 through the 5′ end of exon 3, manifests not only in severe reproductive defects but also in increased fat accumulation in the intestine. Moreover, embodiments disclosed herein reveal that the multiple genetic components that participate in lipid metabolism pathways are deregulated in the absence of klf-3 function. Taken together, the pleiotropic nature of the klf-3 mutation suggests a key physiological role of klf-3 in the regulation of fat metabolism in C. elegans and sheds light on its human counterpart in disease-gene association.

Type 2 diabetes (T2D) is a systemic disease involving changes in both conserved cores (pathway/network of glucose/lipid metabolism) and adaptive conduits for nutrient (food) intake, storage and sensing. In full-blown T2D, insulin resistance and β-cell failure arise owing to chronic pathogenic insults to metabolic networks and enduring perturbations of energy homeostasis. As described above, the family of KLFs has been implicated in the regulation of adipogenesis. In C. elegans, KLF members have essential functions required for metabolic homeostasis: they not only regulate fat storage but intersect insulin signaling. Klf-3 mutation also disrupts its regulatory roles and underlies the chronic pathologic effects of fat accumulation on the endocrine function of intestine.

Embodiments disclosed herein investigated the conserved role of worm klf-3 in adipogenesis using a cellular model. The mouse 3T3-L1 line of preadipocytes is a useful cellular model for studying adipocyte differentiation and roles of various factors in its induction. Based on the successful transfection of worm klf-3 into these cells, this ex vivo system was used as a heterologous model to explore its conserved regulatory role. Stable and inducible lines of mouse 3T3-L1 preadipocyte cells using wild type klf-3 constructs under the direction of a mammalian promoter were established. Given the expression profiling of mammalian KLF genes in these cells and the finding that over-expression of worm klf-3 results in down-regulation of endogenous adipogenic factors upon induction, the role of worm klf-3 in adipocyte differentiation was examined. The effects of klf-3 gene on fat deposition through their over-expression upon induction and during adipocyte maturation were compared. Over expression of worm klf-3 in mouse 3T3-L1 significantly suppresses cell differentiation processes. This allows one not only to mitigate the genetic redundancy of mammalian KLFs but to relate the physiological function(s) of worm KLFs to the conservation of mammalian regulatory networks.

The identification of worm KLFs as an important negative regulator of fat storage in a genetically tractable experimental model will also allow the pursuit of a more comprehensive approach to understand fat biology in humans. Elucidating genes interacting with and mediating KLFs can determine the underlying causes of obesity and associated metabolic disorders like type 2 diabetes (T2D).

Therefore, disclosed herein are methods of regulation of fat deposition comprising upregulating (stimulating or potentiating) or downregulating (suppressing or inhibiting) the activity of at least one KLF. KLFs can be from C. elegans or mammalian sources. In one embodiment, the mammalian KLFs are human KLFs. C. elegans KLFs are selected from the group consisting of klf-1 (NCBI Accession No. NP_{—}497632; SEQ ID NO:1), klf-2 (NCBI Accession No. NP_{—}507995; SEQ ID NO:2) and klf-3 (Wormbase Accession No. WP:CE42120; SEQ ID NO:3) Human KLFs are selected from the group consisting of klf-1 (NCBI Accession No. AAH33580; SEQ ID NO:4), klf-2 (NCBI Accession No. EAW84541; SEQ ID NO:5), klf-3 (NCBI Accession No. NP_{—}057615; SEQ ID NO:6), klf-4 (NCBI Accession No. ABG25917; SEQ ID NO:7), klf-5 (NCBI Accession No. Q13887; SEQ ID NO:8), klf-6 (isoform A:NCBI Accession No. NP_{—}001291, SEQ ID NO:9; isoform B: NCBI Accession No. NP_{—}001153596; SEQ ID NO:10; isoform C: NCBI Accession No. NP_{—}001153597; SEQ ID NO:11), klf-7 (NCBI Accession No. NP_{—}003700; SEQ ID NO:12), klf-8 (isoform 1: NCBI Accession No. NP_{—}009181, SEQ ID NO:13; isoform 2: NCBI Accession No. NP_{—}001152768, SEQ ID NO:14), klf-9 (NCBI Accession No. NP_{—}001197; SEQ ID NO:15) klf-10 (isoform a: NCBI Accession No. NP_{—}005646, SEQ ID NO:16; isoform b: NCBI Accession No. NP_{—}001027453; SEQ ID NO:17), klf-11 (NCBI Accession No. NP_{—}003588; SEQ ID NO:18), klf-12 (NCBI Accession No. NP_{—}009180; SEQ ID NO:19), klf-13 (NCBI Accession No. NP_{—}057079; SEQ ID NO:20), klf-14 (NCBI Accession No. NP_{—}619638; SEQ ID NO:21), klf-15 (NCBI Accession No. NP_{—}054798; SEQ ID NO:22), klf-16 (isoform CRA_a: NCBI Accession No. EAW69448; SEQ ID NO:23), and klf-17 (NCBI Accession No. AAH49844; SEQ ID NO:24) and conservatively modified variants thereof.

As used herein the term “conservatively modified variants” refers to variant peptides which have the same or similar biological activity of the original peptides. For example, conservative amino acid changes may be made, which although they alter the primary sequence of the protein or peptide, do not alter its function. Conservative amino acid substitutions typically include substitutions within the following groups: glycine and alanine; valine, isoleucine, and leucine; aspartic acid and glutamic acid; asparagine and glutamine; serine and threonine; lysine and arginine; phenylalanine and tyrosine.

As used herein, amino acid sequences which are substantially the same typically share more than 95% amino acid identity. It is recognized, however, that proteins (and DNA or mRNA encoding such proteins) containing less than the above-described level of homology arising as splice variants or that are modified by conservative amino acid substitutions (or substitution of degenerate codons) are contemplated to be within the scope of the present disclosure. As readily recognized by those of skill in the art, various ways have been devised to align sequences for comparison, e.g., Blosum 62 scoring matrix, as described by Henikoff and Henikoff in Proc. Natl. Acad Sci. USA 89:10915 (1992). Algorithms conveniently employed for this purpose are widely available (see, for example, Needleman and Wunsch in J. Mol. Bio. 48:443 (1970). Therefore, disclosed herein are amino acid and nucleic acid sequences 85%, 90%, 95%, 98%, 99% or 100% identical to any of the KLF proteins or genes disclosed herein, respectively.

In one embodiment, the method comprises the use of a composition to potentiate or inhibit the expression of at least one KLF gene in a mammal. In another embodiment, the composition includes, but is not limited to, DNA, RNA, cDNA, siRNA, or shRNA.

In another embodiment, the method comprises the use of a composition to potentiate or inhibit the activity of at least one KLF protein in a mammal. In another embodiment, the composition includes, but is not limited to, monoclonal antibodies, polyclonal antibodies, peptides, proteins, and small molecules. In another embodiment, the composition is an agonist or an antagonist.

Disclosed herein are methods and compositions for regulation of fat storage and deposition. The methods and compositions disclosed herein are useful for treating obesity and other fat storage diseases including, but not limited to, lipodystrophy. The methods and compositions disclosed herein are useful in mammals, including humans.

### EXAMPLES

### Example 1

### KLF-1

Materials and Methods

Nematode strains and culture conditions. C. elegans strains were propagated at 20° C. on small petri plates containing nematode growth medium (NGM) and seeded with the E. coli strain OP50. The wild-type strain N2 (Bristol) was used to create transgenic lines.

Relative abundance of Ce-klf-1 mRNAs during worm development. Real-time PCR was used to obtain a stage-specific expression profile of Ce-klf-1 in embryos, staged larvae, and adult worms. To prepare a synchronous population of all developmental stages, embryos were obtained by treatment of gravid hermaphrodites with sodium hypochlorite, then embryos were hatched in water overnight to obtain first-stage larvae (L1). The arrested L1 were transferred onto NCM agarose plates seeded with OP50 bacteria, which allowed the L1 larvae to develop into L2, L3, L4, and adult worm over 40 h. Total RNA was isolated from embryos, larvae, and adult worms using Trizol™ reagent (Gibco BRL). The cDNA was generated from 1 μg of total RNA using SuperScriptR III First-strand synthesis system for RT-PCR (Invitrogen). The Ce-klf-1 specific cDNA was then amplified using forward 5′-GCCACGTCATCACGGGAACC-3′ (SEQ ID NO:25) and reverse 5′-CTCCGAGAGCTGTCGTCGGT-3′ (SEQ ID NO:26) primers by real-time PCR using QuantiTech™ SYBR Green PCR kit (Qiagen) in a 50 μL volume reaction. A second set of primers, forward 5′-GCATTGTCTCACGCGTTCAG-3′ (SEQ ID NO:27) and reverse 5′-TTCTTCCTTCTCCGCTGCTC-3′ (SEQ ID NO:28), were used for amplification of an internal control, the ama-1 transcript. An ABI Prism 7700 Sequence Detector (Applied Biosystems) was programmed for 2 min at 50° C., 15 min at 95° C., followed by 40 cycles of 15 sec at 94° C., 30 sec at 64° C., and 45 sec at 72° C. for both ama-1 and Ce-klf-1. The specificity of PCR amplicon was confirmed on an agarose gel, and the level of each transcript within the stage-specific cDNA preparations was calculated by the comparative Ct method (ABI Prism 7700 Sequence Detection System; Applied Biosystems). The relative content of the transcript corresponding to Ce-klf-1 is expressed as the ratio relative to ama-1. Each experimental point was repeated twice.

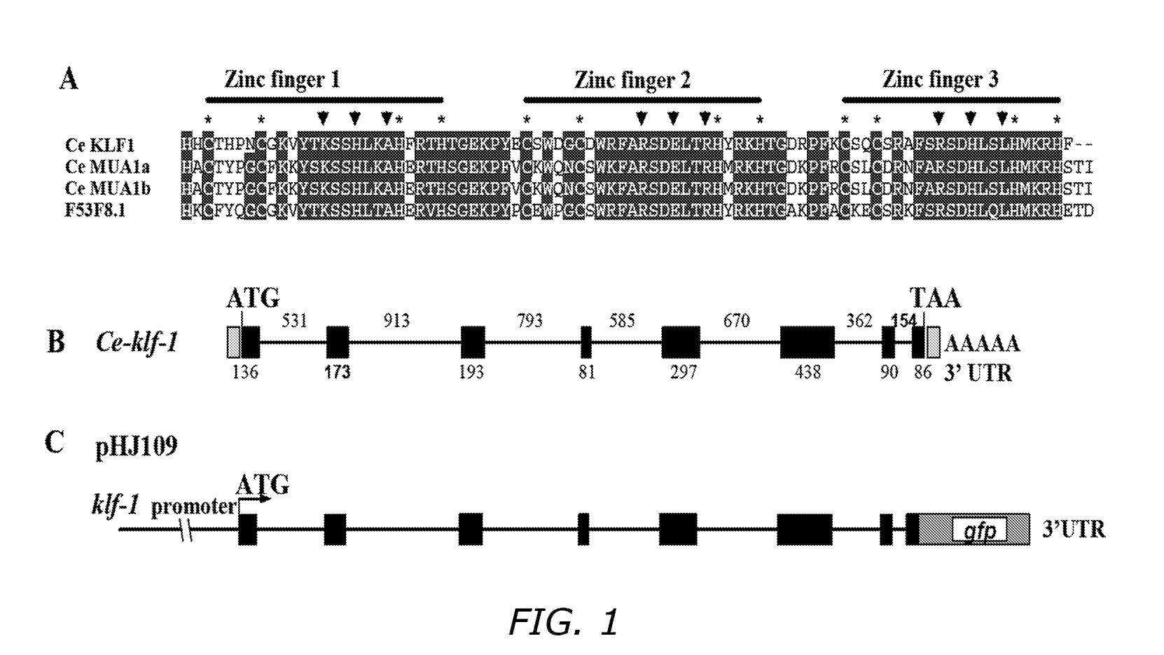

Expression of Ce-klf-1 in vivo. To investigate the expression of Ce-klf-1 in vivo, C. elegans transgenes were created. A translational klf-1::gfp reporter fusion construct (pHJ109; FIG. 1C) that contained 2 kb of the Ce-klf-1 promoter region was made. The coding sequences covered all eight exons in order to achieve its endogenous pattern of gene expression. This 5-kb fragment was PCR amplified using C. elegans genomic DNA as a template and cloned into C. elegans expression vector (pPD95.75) containing the gfp reporter gene. The plasmid DNA was prepared and injected into the gonadal syncytium of individual C. elegans adult hermaphrodites at a concentration of 50 ng/μL. A plasmid DNA (pRF4) containing the dominant selectable marker gene rol-6 (su1006), which encodes a mutant collagen was also coinjected (˜80 ng/μL) with the reporter constructs. C. elegans expressing the rol-6 gene continuously roll over, thereby providing a visible phenotype for selection of transgenic worms. The transgenic C. elegans worms expressing gfp were observed and photographed using Axloskop 2 plus fluorescent microscope (Zeiss) using appropriate filter sets (magnification×400). At least three independent lines were examined for each construct.

Double-stranded RNA preparation and RNAi. RNAi was performed by soaking synchronized L1, L2, L3, and L4 larvae in dsRNA. The full-length Ce-klf-1 cDNA was used as the template for RNA synthesis, and the dsRNA was prepared as described in Hashmi et al. (The Caenorhabditis elegans cathepsin Z-like cysteine protease, Ce-CPZ-1 has a multifunctional role during the worms' development, J. Biol. Chem. (2004), 279, 6035-6045), the methods of which regarding dsRNA preparation are incorporated by reference herein. In brief, cDNA was first cloned into vector pCR 4-TOPO and amplified with commercially available M13F and M13R primers (Invitrogen). Then T3 or 17 RNA polymerase was used for single-stranded sense and antisense RNA synthesis using the MEGAscript high-yield transcription kit (Ambion). For the RNAi experiments, 35-40 synchronized larvae of each developmental stage were separately soaked in 20 μL of 1×PBS containing Ce-klf-1 dsRNA (final concentration of 3 ng/μL) and incubated at 16° C., while another set of 35-40 larvae with the same treatment were kept at room temperature (20-22° C.). After 24 h of soaking, the larvae were transferred to individual E. coli plates, and their development was monitored for 4-5 days under light microscope and photographed using differential interference contrast microscopy (DIC) optics (magnification×400).

Acridine orange assay. To identify the cell corpses, the RNAi worms were stained with acridine orange (AO), an acidophilic dye that stains apoptotic cells in C. elegans. Thus, a positive AO staining could indicate an increase in the number of cell deaths. RNAi worms with a similar age to wild-type (N2) worms were compared. For AO staining, 2 μL of AO stock (10 mg/mL; Molecular Probes, A3568) per mL of M9 buffer was used as the staining solution. Then 500 μl was added and evenly distributed onto a 60-mm NGM plate seeded with E. coli OP50, which contained ˜25 non-starved adult RNAi worms. Similar processes were performed on adult wild-type worms. After 1 hr incubation at room temperature in the dark, both wild-type and RNAi worms were collected separately in tubes. Then worms were washed three times with M9 buffer and transferred to NGM plate (without AO). After another 1 hr incubation in the dark, worms were mounted on the slides and observed under confocal microscopy (Zeiss) at 488-nm wavelength.

Fat staining. To monitor the accumulation of fat contents, RNAi worms were stained with Sudan black, according to Kimura et al. (daf-2, an insulin receptor-like gene that regulates longevity and diapause in Caenorhabditis elegans, Science (1997), 277, 942-946) which is incorporated by reference herein for methods involving Sudan black staining procedures with some modifications. Sudan black is a staining dye that stains fat contents. For Sudan black staining, the nonstarved L4 or adult RNAi worms were fixed in 1% paraformaldehyde in PBS, frozen at −70° C. for 30 min to overnight, and washed. Then worms were incubated in 1 mL of Sudan black (Sigma) solution (0.02% final concentration) in propylene glycol overnight. After incubation, solution was removed and the samples were washed 2× with propylene glycol. Similarly, wild-type (N2) worms of similar age were stained with the same concentration of dye for comparison. The stained worms were mounted on a slide and observed under light microscope equipped with DIC optics.

Results

The gene F56F11.3 was the first gene to be characterized as a C. elegans KLF. Thus it is referred to as Ce-klf-1 (Wormbase; http://www.wormbase.org/). The Ce-klf-1 consists of eight exons (FIG. 1B) encoding a protein product of 497 residues (57.9 kDa, pl 6.2). The size of introns in klf-1 (155-562 bp) is larger than most C. elegans genes that have their introns in the range of 55- to 60-bp long. The Ce-klf-1 also contains three C₂H₂ zinc finger domains in its C-terminal that is the characteristic feature of members of the KLF family (FIG. 1A).

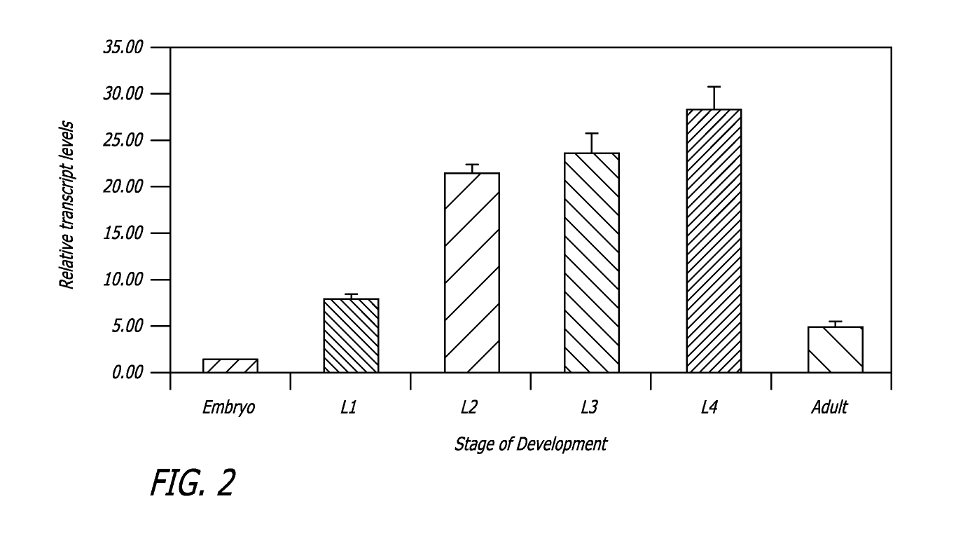

The transcript corresponding to Ce-klf-1 gene is expressed in all developmental stages of the worm and is elevated in larval stages. To understand the Ce-klf-1 expression profile during worm development, RT-PCR analysis was used. Ce-klf-1 is expressed throughout the lifespan of the worm (FIG. 2). However, the distribution of its transcripts vary with development. For instance, the total amounts of gene transcripts were low in the developing embryos relative to a high level of transcripts that were present in the larval stages. The level of transcripts began to increase at L1, remained at an elevated level during all larval development but reached a maximum at L4, followed by a decrease in transcript level in the adult worm (FIG. 2). This pattern of expression suggests that temporal activity of Ce-klf-1 is critical during larval development.

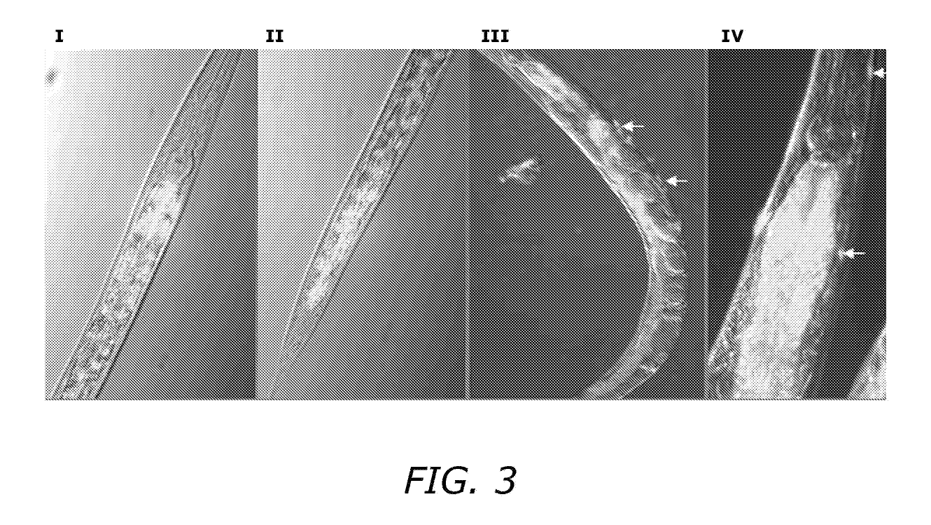

In vivo site of klf-1 expression. Following germline microinjection, injected DNA normally recombines to form a large, extrachromosomal array that is transmitted to the progeny. Because a single transformant may exhibit mosaic patterns of expression, the staining pattern of many transformants derived from at least three independent lines were examined. Interestingly, all transgenic lines exhibited similar patterns of gfp expression. Because a translational reporter gene can provide information at the subcellular localization of the endogenous gene product, transgenic lines that expressed a translational fusion construct (pHJ109) were created; in these transgenic lines, the expression of gfp was absent from embryos, but present in the intestine of all larval stages and adult worm (FIG. 3, I and II). The gfp expression was also prominent in a few neuronal and hypodermal cells (FIG. 3, III and IV). Although the potential role of Ce-klf-1 in neuronal and hypodermal cells remains to be determined, the presence of high-level expression of gfp in intestine points to a role of this gene in fat regulation.

The C. elegans klf-1 level affects egg laying. In C. elegans, egg laying occurs through a simple motor program that involves specialized smooth muscle cells. The contraction of these muscles allows the vulva to open and at the same time compresses the uterus, resulting in egg laying. To gain an insight into the function and localization of Ce-klf-1, transgenic lines carrying extra copies of this gene were generated. As discussed above, the plasmid pHJ109 contains the entire coding sequence including the 2 kb of 5′ upstream sequence from its ATG. The construct was microinjected at three different concentrations 50, 25, and 10 ng/μL. At the highest concentration (50 ng/L), less than 10 F1 progeny were produced from each injected hermaphrodite but none of those F1 progeny contained the transgene, suggesting that Ce-klf-1 is toxic at high concentrations. However, at 25 ng/μL concentration, ˜70 F1 transgenic progeny were produced but only few of them were able to reach adulthood.

Those adult transgenic worms produced many eggs, which were accumulated in the uterus and only three to five eggs were laid. This phenotype was only observed in those F1 worms that expressed both roller and gfp reporter markers. The F1 roller transgenes obtained from injection of pRF4 plasmid alone were as normal as wild type. The injection of much lower concentration (10 ng/μL) of plasmid enabled the establishment of stable lines of transgenic worm. The patterns of gfp expression in these stable lines were similar to those obtained with a higher (25 ng/μL) concentration. The egg-laying defect was likely due to the presence of extra copies of the Ce-klf-1, because the worm had no defects when injected at a lower concentration of plasmid. The presence of Ce-klf-1 gene sequences in the transgenic worm was also confirmed by PCR using primers covering the inserted sequences in pHJ109 construct.

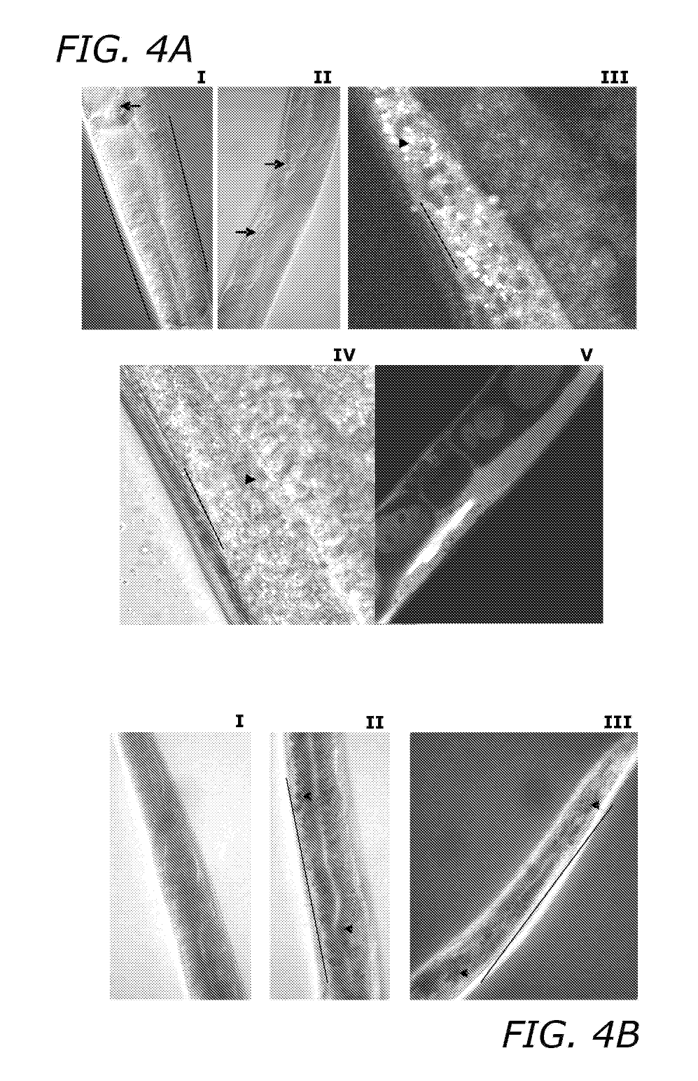

Ce-klf-1 is involved in increased cell death and phagocytosis. The RNAi assays were performed on various stages of worm larvae by soaking them in dsRNA. This soaking technique allows easy administration of dsRNA to many worms at once, and is particularly effective for larvae. The larvae soaked in dsRNA appeared to be healthy, completed their four stages of larval development and reached adulthood. Each RNAi egg-laying hermaphrodite (n=35) produced on average 69 viable embryos over a 5-day period. In comparison, the wild-type adult hermaphrodites produced ˜257 embryos in the same period. No delays in larval development were observed, and movement of RNAi animals was normal, indicating that there were no obvious abnormalities in the nervous system or muscle development. In all worms, cells of the intestine, hypodermis, and pharynx appeared to be normal. The spermatheca was present in most worms as indicated by DAFT (4′,6-diamidino-2-phenylindole dihydrochloride) staining (data not shown). However upon careful observation of the RNAi-treated egg-laying hermaphrodites (n=35), it was found that those hermaphrodites contained many dead cells in the uterus as well as in the germline. Morphologically, those cells were similar to apoptotic cells (FIG. 4A, II and IV). The RNAi animals were stained with the vital dye AO, which stained many cells (FIG. 4A, III) of the germline, indicating that the dead cells were apoptotic.

AO staining was barely observed in wild-type worms (FIG. 4A, V). As positive control, larvae were soaked in cpz-1 dsRNA that conferred a molting defect phenotype. For negative controls, the L1/L2 larvae were soaked in buffer or in unrelated dsRNA. These larvae continued their normal growth and development (FIG. 4A, I).

Suppression of Ce-klf-1 increased fat contents in the intestine. Because the intestinal expression of Ce-klf-1 is similar to worm genes known to be involved in fat metabolism, it was evaluated whether Ce-klf-1 has a role in the regulation of fat storage. Sudan black was used to stain fat contents in the intestine of the RNAi worm and compared its staining with wild-type worms. Extensive accumulation of fat in the intestine of RNAi worms compared to a very low accumulation in wild-type worms (FIG. 4B, I-III) was found, suggesting that Ce-klf-1 has an essential function in fat regulation.

### Discussion

Krüppel-like transcription factors are important regulators of cellular development and differentiation. In the described study, Ce-klf-1a previously uncharacterized C₂H₂ zinc finger domain protein in C. elegans, was characterized. Ce-klf-1 was shown to be essential in fat regulation. Ce-klf-1 RNAi results show an increase in fat storage in the affected worm, suggesting that loss of function of this gene disturbs normal fat metabolism and thus increases fat storage. The altered fat storage in the RNAi worms is also consistent with its expression in the intestine, and a steady increase in Ce-klf-1 levels during larval development. The intestine is the major site for fat metabolism in C. elegans. Thus the localized expression of Ce-klf-1 in the intestine of larvae and adult worms is likely to be important with regard to its essential regulatory role in fat regulation. Following RNAi, a low production of progeny and a pronounced accumulation of apoptotic cells in older egg-laying hermaphrodites was also observed, suggesting that suppression of Ce-klf-1 increased cell death. Ce-klf-1 is not required for embryonic or germline development because germline, oocytes, and spermatheca appeared to be normal in RNAi worms. However, transgenic egg-laying hermaphrodites over-expressing Ce-klf-1 in the intestine developed egg-laying defects.

Several C. elegans genes function in programmed cell death. For example, the genes ced-3 and ced-4 are required for cell death, while ced-9 protects cells from programmed cell death. Thus suppression of ced-9 function results in death of many cells that normally survive. The dead cells are engulfed by other neighboring cells for phagocytosis. In C. elegans, the engulfment of dead cell is controlled by a group of ced, or cell death genes. In addition, ced-1, ced-2, ced-5, ced-6, ced-7, and ced-10 are involved in phagocytosis, and mutation in any of these genes results in the accumulation of many dead cells in the germline. Data presented here suggests that suppression of Ce-klf expression increased cell death. It is possible that the dead cells were not engulfed or removed by phagocytosis in the Ce-klf-1 RNAi worm. The two mutant alleles of klf-1 (tm731, a 343-bp deletion, and tm1110, a 491-bp deletion) that have a short deletion in their intronic sequence show a wild-type phenotype. A tm731/tm1110 double mutant was also created, which also showed a wild-type phenotype indicating that Ce-klf-1-null mutant is needed to determine the nature of Ce-klf-1 function in cell death and phagocytosis.

In summary, the data demonstrates the essential role of C. elegans KLF in the regulation of fat storage. Suppression of Ce-klf-1 function results in significant accumulation of fat that directly or indirectly causes reproductive defects in the RNAi hermaphrodite. High-level expression of Ce-klf-1 during larval development as well as its localization in the intestine, supports its role in these processes. The identification of worm KLFs as an important regulator in fat storage allows pursuit of a comprehensive approach to understand fat-linked metabolic disorders.

### Example 2

### KLF-3

Materials and Methods

Nematode strains and culture conditions. All C. elegans strains used in this study were maintained and propagated at 20° C. on small petri plates containing nematode growth medium (NGM) seeded with E. coli OP50. The WT strain N2 (Bristol) was used to create transgenic lines. The homozygous klf-3 (ok1975 and rh160) mutant alleles were obtained from C. elegans Genetics Center (Minneapolis, Minn.).

Stage-specific profile of the Ce-klf-3 mRNA transcript. Real-time quantitative RT-PCR (qRT-PCR) was used to profile the stage-specific expression of klf-3 in embryos, staged larvae, and adult worms. A synchronous population of all developmental stages was prepared as previously described. Embryos were obtained by treating gravid hermaphrodites with sodium hypochlorite, and then hatched in water overnight to derive L1 larvae. The arrested L1 larvae were transferred onto nematode growth media (NGM) plates, and allowed to develop into L2, L3, L4, and adult worms over 40 hrs. Total RNA was prepared from those worms using Trizol™ reagent according to the manufacturer's protocol. The klf-3 cDNA was prepared from 2 μg of total RNA in a 50 μl volume reaction with forward 5′-CCACTACATCAAGCGAGC-3′ (SEQ ID NO:29) and reverse 5′-GCGCTTCATGTGAAGACT-3′ (SEQ ID NO:30) primer using qRT-PCR and QuantiTechn™ SYBR Green PCR kit (Qiagen). Another set of primers, forward 5′-GCATTGTCTCACGCGTTCAG-3′ (SEQ ID NO:27) and reverse 5′-TTCTTCCTTCTCCGCTGCTC-3′ (SEQ ID NO:28), was used to amplify internal control, ama-1 transcripts. An ABI Prism 7700 Sequence Detector was programmed for an initial step of 2 min at 50° C., 15 min at 95° C., followed by 40 cycles of 15 sec at 94° C., 30 sec at 58° C., 45 sec at 72° C. The specificity of each amplicon was confirmed on agarose gel electrophoresis and the relative level of each transcript within a stage-specific cDNA preparation was calculated by the comparative Ct method. The relative abundance of the transcript is presented as the ratio between klf-3 and ama-1.

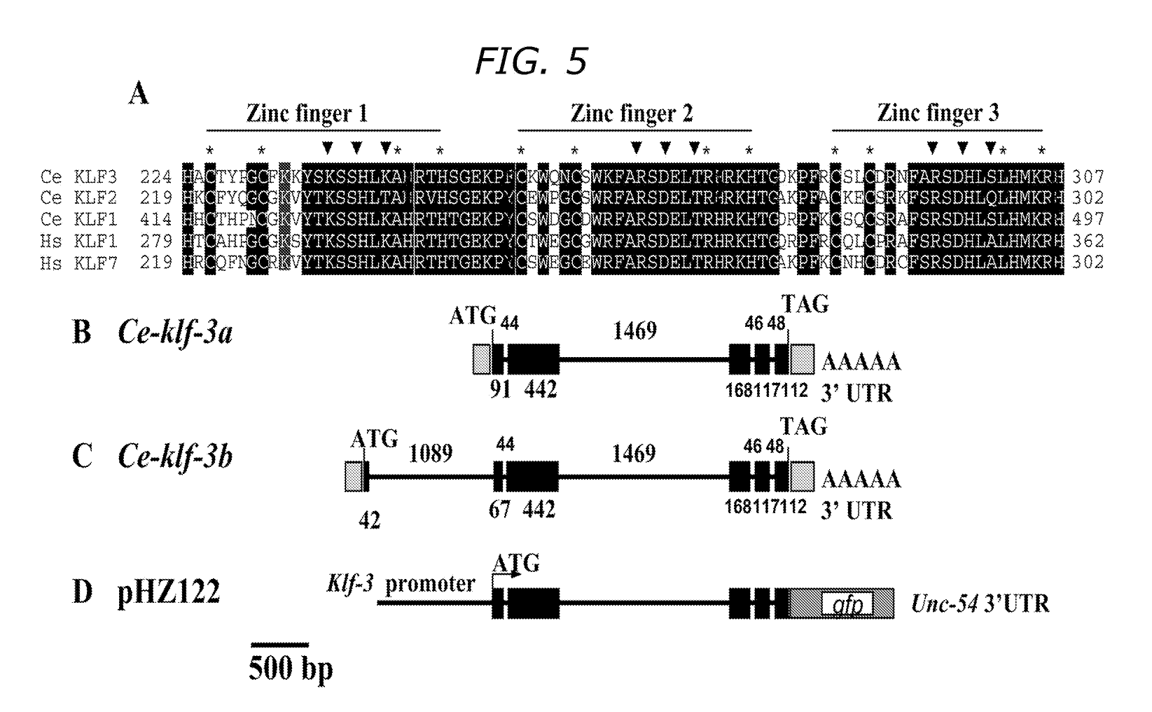

In vivo site of klf-3 expression. To determine the in vivo expression site of klf-3, a klf-3::gfp translational fusion reporter construct that contained the 5′ putative promoter and the entire coding sequence covering its five exons was made. This sequence was PCR amplified from WT DNA, digested with restriction enzymes, and cloned into the gfp reporter vector pPD95.75. The resulting construct pHZ122 (FIG. 5) was sequenced to confirm the WT sequence and correct fusion of klf-3::gfp. The pHZ122 plasmid was prepared using the Concert™ rapid plasmid miniprep system (Invitrogen), and then injected into the gonadal syntium of individual adult hermaphrodites at a concentration of 50 ng/μl. The pRF4 plasmid, which contains the dominant marker rol-6 (su1006) encoding a mutant collagen, was co-injected (80 ng/μl) to confer a visible roller phenotype to transgenic worms. The F3 roller worms were selected for observing klf-3::gfp expression. At least three independent transgenic lines were examined for each construct.

Characterization of klf-3 (ok1975) and klf-3 (rh160) mutant alleles. To determine the location and size of mutation in klf-3 (ok1975) and klf-3 (rh160) mutant alleles, genomic DNA was prepared from homozygous worms of each strain. A series of primers specific to either klf-3 or its flanking genes was designed to amplify the above genomic DNA by PCR. The PCR products were then cloned into TOPO cloning vector and sequenced to pinpoint the exact breakpoints of gene deletion. To determine the effect of the mutation, RT-PCR, cloning, and sequencing were performed to characterize transcript expression of the affected genes in klf-3 (ok1975) and klf-3 (rh160) genetic backgrounds.

Genetic and phenotypic analyses of klf-3 (ok1975) mutant. To dissect the loss-of-function of klf-3 (ok1975) mutant allele, the mutant strain was backcrossed three times using WT males according to a standard protocol and maintained as homozygous. The presence of deletion after each crossing was confirmed by single-worm PCR and DNA sequencing. Individual homozygous mutant hermaphrodites were grown on plates at 20±1° C. and their self-progeny used for experimentation. For morphological comparison between WT and mutants, living animals were observed under a microscope using Nomarski differential interference contrast microscopy. To measure fertility, L1/L2 larvae were individually laid onto NGM plates seeded with E. coli OP50 bacteria, and their growth and development was observed at room temperature. When these worms began to lay eggs, the number of embryos produced by each of them was counted. Individual worms were transferred to fresh NGM plates every 24 hours followed by counting the eggs and larvae for five consecutive days. If a hermaphrodite worm did not produce any embryos in this period, it was considered sterile. If a hermaphrodite worm produced 40±10 viable embryos, it was considered semi-sterile.

Rescue assays. To rescue the klf-3 (ok1975) mutation by complementation, the transgenic line expressing the klf-3::gfp (pHZ122) translational fusion construct was used to confirm that the phenotype due to deletion was only due to the knockdown of the klf-3 gene. The procedure to rescue deletion mutant worms using transgenic strains followed the procedures described by Janke et al. (Interpreting a sequenced genomic: Towards a cosmid transgenic library of Caenorhabditis elegans. Genome Res. 7 (1997) 974-985) and Hashmi et al. (The Caenorhabditis elegans CPI-2a cystatins-like inhibitor has an essential regulatory role during oogenesis and fertilization. J. Biol. Chem. 281 (2006) 28415-28429), both of which are incorporated by reference herein for procedures related to rescue deletion mutant worms using transgenic strains. In brief, heterozygous mutant males were created by crossing hermaphrodite klf-3 (ok1975) mutant worms with wild type males, 15 individual transgenic hermaphrodites expressing the rescue gene were then crossed, each with 12 heterozygous mutant males. After 24-36 h of mating, the hermaphrodites were transferred individually to fresh NGM plates and allowed to produce progeny. The F1 progeny was screened for males exhibiting the roller phenotype (rol-6) indicating the presence of the transgene within the worms and thus successful crossings. In addition, single worm PCR was performed on the roller worms to ensure that these worms contained the rescue gene. Twenty L4 roller hermaphrodites from a successful mating plate were individually picked and transferred to fresh plates to allow self-fertilization. For this transgene rescue experiment the individual worms were of three possible genotypes: klf-3 (ok1975)/+; klf-3 (Ex) or +/+; klf-3 (Ex): The Ex designates extrachromosomal array for each rescue gene. The worms of these three genotypes were screened for the presence of sterile or non-sterile animals over their reproductive periods. These worms were also tested for fat accumulation. If the worms produced ˜200 progeny during their reproductive periods (usually 5-6 days of their adulthood) and the presence of fat granules in their intestine were comparable to wild type they were considered rescued. The rescue was correlated with the presence or absence of the expression of klf-3 (klf-3::gfp construct) as well as their genotype (the presence and absence of klf-3 deletion) using single worm PCR (fertile and non-fertile animals) with the corresponding gene specific primers. The progenies of the heterozygous fertile or non-fertile roller worms were self-fertilized to obtain homozygotes. Single worm PCR on roller mothers was used to confirm their genotype and then the progenies of homozygous worms were tested for fertility or absence of fat accumulation.

Fat staining and microscopic examination of lipid droplets. Fat-staining was performed with Sudan black. In brief, mixed populations, as well as non-starved L4 or adult klf-3 mutant worms were fixed in 1% paraformaldehyde in PBS separately, frozen at −70° C. for 30 minutes to overnight, washed, and incubated overnight in 1 ml of Sudan black solution (0.02% final concentration in propylene glycol). Then, the samples were washed twice with propylene glycol, mounted on a slide, and observed under light microscope equipped with DIC optics. WT worms of similar age were treated in the same way for comparison. The experiment was repeated twice. Each experiment contained three replicates. Electron microscopic examination of worm thin sections was performed as previously described.

Analysis for fatty acid composition. A synchronized population of young adults of both wild type (N2) and klf-3 (ok1975) mutant worms were grown on NGM plates seeded with E. coli OP50. Then the worms were washed off the plates with water, and rinsed 3 times. The worms were stored at −80° C. Fatty acids extraction and analysis were performed at the Fatty Acid Analysis Laboratory (University of Florida, Gainesville, Fla.). Fatty acids were extracted according to the method described by Sasser (Bacterial identification by gas chromographic analysis of fatty acid methyl esters (GC-FAME) Technical Note #101 (2006) MIDI, Inc., Newark, Del.) and Brock et al. (Genetic regulation of unsaturated fatty acid composition in C. elegans. PLoS Genet. 2 (2006) 997-1005), both of which are incorporated by reference herein for fatty acid extraction methods with several minor modifications. Fatty acids as fatty acid methyl esters (FAMEs) were detected using an Agilent 6890 gas chromatograph with an FED. Fatty acids were identified using the Sherlock Microbial Identification System (MIS) version 4.5 with the EUKARY peak library and method version 3.71 (MIDI, Inc). Peaks are the representative of three measurements from three independent extractions of mutant and wild type nematodes.

Analysis of genes involved in fatty acid metabolism. Because of the high fat accumulation in klf-3 (ok1975) mutants, klf-3 might regulate genes involved in lipid metabolism. To test this premise, 44 genes in the C. elegans genome predicted to participate in fatty acid synthesis, desaturation, elongation and β-oxidation pathways were identified (Table 1). A synchronized adult population of both klf-3 (ok1975) and N2-Bristol (WT) were grown at room temperature (22±1° C.) on NGM plate seeded with E. coli OP50 bacteria and collected by washing off plates in PBS, washed 3× in PBS buffer. As described above, the total RNA was prepared from those worms using TRIZOL™ reagent, and then the cDNA was prepared from 2 μg of total RNA in a 50 μl volume reaction. qRT-PCR was used to measure the expression level of each of forty genes in WT and klf-3 (ok1975) mutant worms with gene-specific primers designed to amplify each of the above genes along with control primers to amplify 18S rRNA, tbb-2 (β-tubulin), and ubc-2 (ubiquitin-conjugating enzyme, E2). The expression of transcripts in WT vs. klf-3 (ok1975) mutants is presented as the mRNA abundance of each gene relative to control genes.

TABLE 1

Genes predicted to participate in fatty acid synthesis,
desaturation, elongation and b-oxidation pathways and
primer pairs to be used in RT-PCR analysis

| | |
|---|---|
| Gene | Primer Pair Sequences |

| | | | | |
|---|---|---|---|---|
| acs-1 | SEQ ID NO: 31 | tatccaccaccaccagtg | SEQ ID NO: 32 | atacatagggtagggggg |
| acs-2 | SEQ ID NO: 33 | atgtcgctgatgctcatgtcg | SEQ ID NO: 34 | cagttccgagacccaacagc |
| acs-3 | SEQ ID NO: 35 | aaatggcttccaaccggc | SEQ ID NO: 36 | tttccgtccaacgccttca |
| acs-11 | SEQ ID NO: 37 | aactgttggcccggctgta | SEQ ID NO: 38 | ctccgacgggactacaattgc |
| cpt-5 | SEQ ID NO: 39 | tcccgcaggaagttattgaaa | SEQ ID NO: 40 | gcttgatttcctccgaatcg |
| dhs-25 | SEQ ID NO: 41 | ctaaatccaccggtaacttcc | SEQ ID NO: 42 | caaggccggagtcatcg |
| ech-1 | SEQ ID NO: 43 | aaccaagaggcggcaaagc | SEQ ID NO: 44 | gttggcatggctcaaattgg |
| ech-8 | SEQ ID NO: 45 | tcaattccttgaagccatcc | SEQ ID NO: 46 | gaacgatcaggatgccgtc |
| ech-9 | SEQ ID NO: 47 | gagcaatcctctcaacggtg | SEQ ID NO: 48 | ccggtgtattgaagaaggtgt |
| elo-2 | SEQ ID NO: 49 | gattctgttcctggttgcgc | SEQ ID NO: 50 | gacatgcccgtaagagtggaa |
| elo-6 | SEQ ID NO: 51 | tcaaggttccagcatggattg | SEQ ID NO: 52 | tcttgccacctcccttgatg |
| fasn-1 | SEQ ID NO: 53 | tctcatccaatctctcccctca | SEQ ID NO: 54 | ttgaaatcaagggtgggcag |
| fat-1 | SEQ ID NO: 55 | acggacacgttgcccatca | SEQ ID NO: 56 | gcctttgccttctcctcgag |
| fat-2 | SEQ ID NO: 57 | attaccaacggtcacgtcgc | SEQ ID NO: 58 | gcctttgcagcctcaactcc |
| fat-3 | SEQ ID NO: 59 | accaacatggccacttcgg | SEQ ID NO: 60 | cattcagattgcaacgtggc |
| fat-4 | SEQ ID NO: 61 | tggaggtttcctgctctctca | SEQ ID NO: 62 | tggtaaaccatttgctgctgc |
| fat-5 | SEQ ID NO: 63 | acgctacatggtgcatcaac | SEQ ID NO: 64 | agccgaacttcttgcactg |
| fat-6 | SEQ ID NO: 65 | ctaccagctcatcttcgaggc | SEQ ID NO: 66 | gatcacgagcccattcgatgac |
| fat-7 | SEQ ID NO: 67 | cgatacttctgtttccgcc | SEQ ID NO: 68 | ttcttgattcttcacttccg |
| pod-2 | SEQ ID NO: 69 | tcggtcgagtttgcggatg | SEQ ID NO: 70 | tcgtccattgagctgttcgg |
| B0272.3 | SEQ ID NO: 71 | ccgtctcttggtgccttaca | SEQ ID NO: 72 | tcggctagcaatcatcattc |
| B0303.3 | SEQ ID NO: 73 | atoggacatocattoggag | SEQ ID NO: 74 | aaggcacgacaaccgcta |
| C1703.4 | SEQ ID NO: 75 | ttctcagcagctggtcattg | SEQ ID NO: 76 | gctccagaagtggcttgca |
| C48B4.1 | SEQ ID NO: 77 | aagttgtttatcgcccgtgg | SEQ ID NO: 78 | atcacggcgaccgagtactg |
| F08A8.1 | SEQ ID NO: 79 | cgtagacatgaccatcacgg | SEQ ID NO: 80 | caagtcatccgtggagttga |
| F08A8.2 | SEQ ID NO: 81 | agcctgccttctagccatg | SEQ ID NO: 82 | ggattgctattggcggatg |
| F38H4.8 | SEQ ID NO: 83 | agcgcgattgcactacgt | SEQ ID NO: 84 | tctgaagctcaaggattgcc |
| F44C4.5 | SEQ ID NO: 85 | ttgcaagtgatctccatccac | SEQ ID NO: 86 | acccgacttacaaacgcaac |
| F53A2.7 | SEQ ID NO: 87 | tacttgacgttgcggcg | SEQ ID NO: 88 | ctgctgtcggttgtgatcc |
| F54C8.1 | SEQ ID NO: 89 | acgagtagaatccgtcaccag | SEQ ID NO: 90 | aggagatgcatcaatgaccg |
| F59F4.1 | SEQ ID NO: 91 | cttcgagatggcacgttcg | SEQ ID NO: 92 | caaccgcatttggacgcat |
| KO5F1.3 | SEQ ID NO: 93 | aagttctggaacagtgtgcg | SEQ ID NO: 94 | tcatgattgcggatatggc |
| R06F6.9 | SEQ ID NO: 95 | tgctgcaatcgcttggtg | SEQ ID NO: 96 | gcttctgagatgctgttggtc |
| R0703.4 | SEQ ID NO: 97 | tgagttggagttgtcaagcc | SEQ ID NO: 98 | ctcgtagcagtcgtcgttcc |
| R07H5.2 | SEQ ID NO: 99 | cgattgagccaaccaactc | SEQ ID NO: 100 | tcggatcgagaaggtgacc |
| RO9E10.3 | SEQ ID NO: 101 | aagcaactggcgtcaagtg | SEQ ID NO: 102 | ttacgttcatggcgatatgg |
| T05G5.6 | SEQ ID NO: 103 | ctcttctcggcaaaagcg | SEQ ID NO: 104 | ccatggaggtgtgccttac |
| T08B2.7 | SEQ ID NO: 105 | tcgcgaagatcaagaagaga | SEQ ID NO: 106 | caatgaggcgcttctatgtc |

Results

The C. elegans genome contains three KLFs. Three KLFs), klf-1 (F56F11.3, klf-2 (F53F8.1) and klf-3 (mua-1 or F54H5.4), in the worm genome (http://www.wormbase.org) were identified. They are genuinely related as all contain three highly conserved C-terminal C₂H₂ zinc fingers: klf-1 and klf-3 are both similar to human klf-1 and klf-2 is very similar to human klf-7 (FIG. 5A), yet they display little homology in their N-terminal regions (not shown). In C. elegans, klf-3 occurs in two isoforms differing in the 5′-coding region: klf-3a has five exons which encode a protein of 309aa, while klf-3b has six exons which encode a protein of 315aa (FIG. 5, B, C). The ATG start codon of klf-3a begins approximately 1 kb downstream of the klf-3b ATG start codon. The spliced EST data available on Wormbase strongly supports that klf-3a and klf-3b use separate promoters. Preliminary data on reporter gene expression also indicates that these two genes show differential gene expression (data not shown). In genome-wide screens, these worm KLFs did not demonstrate any role in fat regulation. However, as described above, klf-1 is involved in fat metabolism; klf-1 RNAi causes increased fat accumulation in the intestine of RNAi worm. This finding prompted the determination of whether klf-3 possesses a similar functional role and whether klf-3 is the same gene mutated and genetically mapped as in mua-1 (muscle attachment abnormal-1).

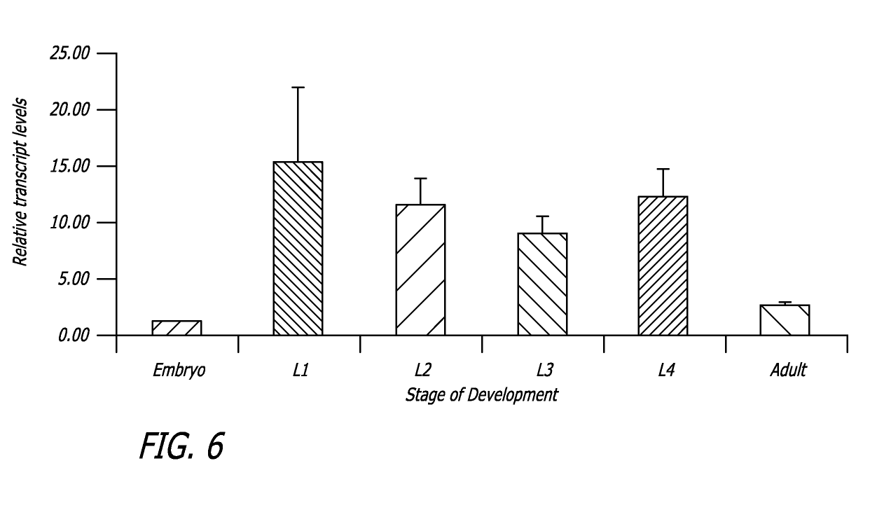

The klf-3 gene is expressed in all stages but is particularly elevated during the larval stages of development. Because the expression of klf-3 has not been well characterized, the stage-specific pattern of its expression during development by measuring its mRNA levels in embryos, larvae, and adults was first determined. Using real-time RT-PCR, a reproducible estimate of the relative abundance of klf-3 transcripts in various developmental stages was obtained (FIG. 6). The results presented indicate that klf-3 is expressed throughout the lifespan of the worm but in varying abundances. The total amount of klf-3 transcripts was lower in embryos and higher in developing larvae. The peak amount was seen in L1 larvae. After L1 growth, the klf-3 level dropped slightly in the L2, L3, and L4 stages (FIG. 6). Since C. elegans increases in size from larval to adult stages after the final molting, these results suggest that the temporal elevation of klf-3 expression in larval stages is critical to the functional activity of klf-3 in these stages of development.

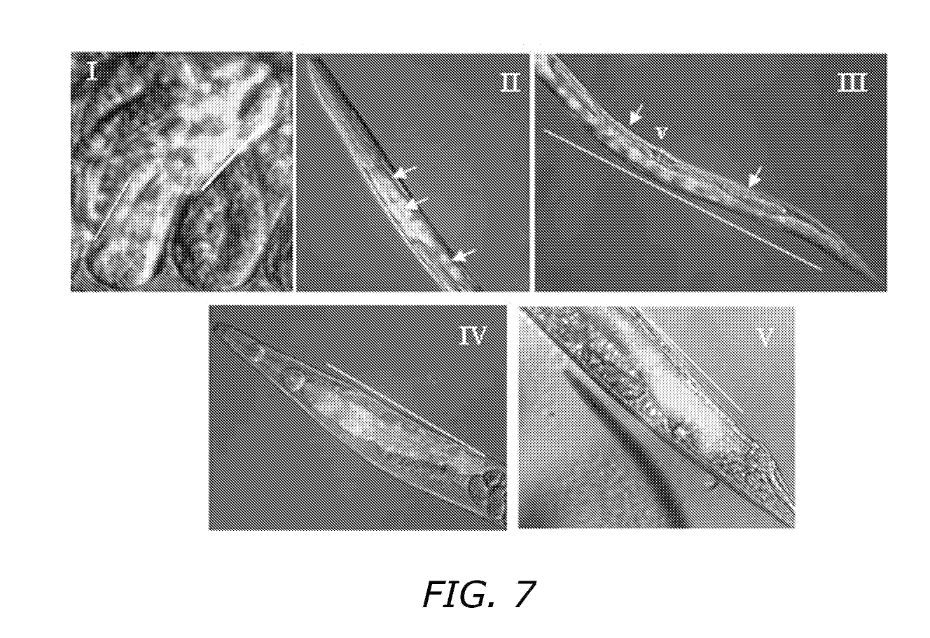

The intestine is the major site of klf-3 gene expression. To determine the timing and location of klf-3 expression in vivo, transgenic lines carrying the klf-3::gfp fusion gene, which was driven by a cognate promoter, a 1.0-kb genomic sequence upstream of the first ATG codon, was established (FIG. 5D). As shown, klf-3::gfp expression first appeared in the early larvae which were still enclosed in the eggshell of the embryo (FIG. 7, I). During larval development, gfp fluorescence was frequently observed in the intestinal cells of developing larvae, young adults, egg-laying hermaphrodites, and male worms (FIG. 7, II-V). Gfp fluorescence was very strong and persisted even in very old adult worms. This pattern was consistently seen in all three transgenic lines. It appears that the expression of klf-3 in the intestine during larval development as well as in adults is genetically programmed, corroborating the mRNA data. These results indicate that the activity of klf-3 is primarily in the intestine, given that the intestine is a major site of fat metabolism, performing many vital functions in C. elegans such as food digestion, nutrient absorption, and energy storage.

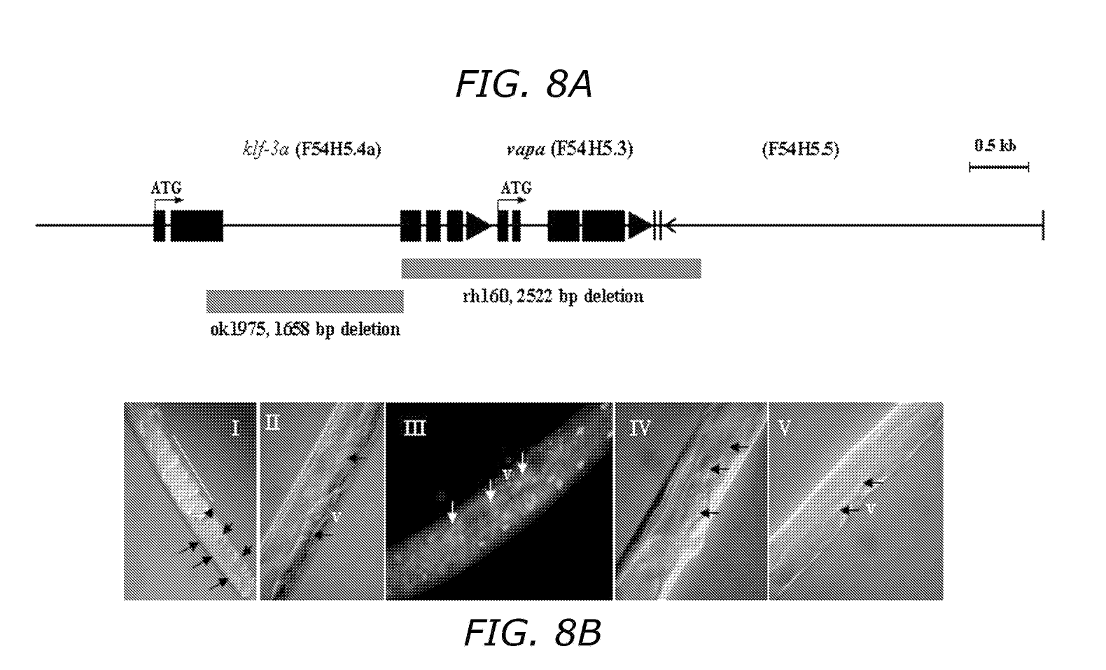

Two alleles of klf-3 mutant exhibit different deletions and phenotypes. Before the phenotypic characterization of klf-3 (ok1975) and klf-3 (rh160) mutant alleles, their genomic abnormalities and expression of transcripts was first determined. It was confirmed in the klf-3 (ok1975) allele a 1658-bp deletion spanning the 3′ end of exon 2 through to the 5′ end of exon 3 of klf-3, establishing that klf-3 is the only gene mutated in this strain (FIG. 8). In contrast, a 2.5-kb deletion in the klf-3 (rh16O) allele, which covers three genes from exon 3 in klf-3, and to F54H5.3 and the 5′ end of an uncharacterized F54H5.5 gene was identified (FIG. 8) suggesting that an extensive genetic disruption in this mutant worm has occurred that affects other neighboring genes in addition to klf-3. The C. elegans wormbase indicates that F54H5.3 encodes a VAMP-associated protein; its RNAi causes a reduction in fat content and abnormal lipid metabolism. Although both klf-3 (ok1975) and klf-3 (rh160) alleles are loss-of-function mutants, the phenotype of the klf-3 (ok1975) allele is not as severe as the klf-3 (rh160) allele in terms of survival, growth, development and movement. This is consistent with the finding that the latter carries a multi-gene deletion. Here, the functional and phenotypic alterations in the klf-3 (ok1975) mutant was the focus because the deletion there affects the klf-3 gene only and provides an advantage for genetic and phenotypic analyses by establishing the baseline of loss-of-function through a single gene alteration.

The klf-3 mutant worms manifest abnormal morphology and severe reproductive defects. To determine the morphology and phenotype of klf-3 (ok1975) mutant worms, the growth and development of L1 larvae were observed by growing them individually on NGM plates. These L1 worms were able to grow to adulthood without obvious defects in movement, pharyngeal pumping, intestinal contraction, or morphology; however, they gradually became sick. In a batch of 40 worms, 12 (30%) developed into sterile adults. In the adult stage, these sterile worms moved slowly and their intestines appeared very dark, despite an apparently normal lifespan. The remaining 28 mutant adult hermaphrodites (70%) each produced 40±10 (mean±standard error) viable offspring over 5 days before becoming sterile. In comparison a WT hermaphrodite produced 262±12 viable embryos in the same period. Based on the two distinctive phenotypes, those 12 and 28 worms were classified as sterile (no progeny) and semi-sterile (reduced progeny), respectively.

After 5 days of observing their reproductive behaviors, both types of mutant hermaphrodites were transferred to a slide for further microscopic examination. Besides reproductive defects, various structural changes in live mutant worms were found. To visualize the nuclei of germ cells or developing oocytes, worms were fixed and stained with DAPI. The typical patterns were seen in the germline and oocyte areas of normal worms but not in sterile mutants, where morphologically abnormal oocytes and disorganized gonads were evident (FIG. 8, II). In addition, few cells showed DAPI staining around the vulva (FIG. 8, III). The acridine orange (AO) staining was negative in the germline area indicating that the morphologically abnormal cells were not apoptotic (data not shown). The oocyte region of the gonad arm was filled with small morphologically abnormal oocytes. The worms were fat in appearance with darkened intestines and degenerated gonads. In L4 and early adult semi-sterile worms, germ cells and oocytes appeared normal. The semi-sterile worms also appeared normal in fertilization and egg-laying, but their oogenesis became impaired after 40-50 oocyte-sperm fusion events. The degeneration of embryos began with the appearance of disorganized clumps of dead cells in the uterus (FIG. 8, IV). In some older egg-laying worms, gonadal muscle was also detached (FIG. 8, V). The gradual appearance of egg-laying defects in the semi-sterile mutant worms could be due to the gradual deterioration of certain klf-3 related activities.

Mutant Rescue. In order to validate that the reproductive defects and fat accumulation observed in klf-3 (ok1975) mutant worms is due to the deletion of the klf-3 gene, the klf-3::gfp (pHZ122) fusion genes bearing full klf-3 protein-coding segments for the rescue of different aspects of the klf-3 (ok1975) mutant phenotype were tested. There are three phenotypes of klf-3 (ok1975) mutant: complete sterility (0 progeny), semi-sterility (˜50 progeny) and excessive fat accumulation. It was found that the pHZ122 construct can direct expression of klf-3 proteins to rescue major aspects of the klf-3 loss-of-function mutant phenotype. It was found that this construct was able to rescue the semi-sterile phenotype in 25 (n=30) klf-3 (ok1975); klf-3::gfpEx transgenic worms. The rescued worms were fertile and produced on average 215±8 viable progeny during their reproductive period (4-5 days of adulthood) and were positive for klf-3 (Ex) as indicated by gfp expression, whereas 5 (n=28) klf-3 (ok1975); klf-3::gfpEx remained completely sterile (0 progeny) during their reproductive period. Although the expression levels of klf-3::gfp fusion genes were high in multiple transgenic lines tested, this expression level may not be sufficient to rescue the complete sterility of the klf-3 mutant. Alternatively, there are other factors involved that cause complete sterility in the mutant worms. Fat accumulation in klf-3 (ok1975); klf-3::gfpEx transgenic animals by Sudan black staining was also observed. These worms displayed significantly lower fat content than klf-3 (ok1975) mutants. The results clearly indicate that the reproductive defect and excessive fat build up in the klf-3 (ok1975) mutant worm was the result of a disruption in the normal function of klf-3.

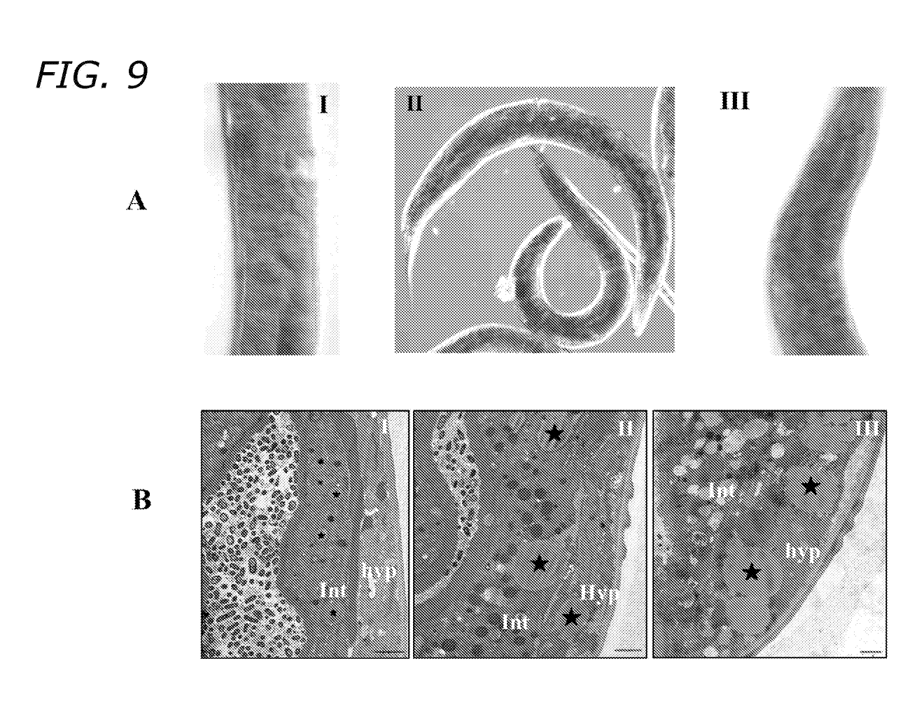

The klf-3 mutant worms accumulate abnormally high fat contents. Given the intestinal expression of klf-3 and the appearance of fat in klf-3 (ok1975) mutants, whether the klf-3 deletion caused the fat accumulation phenotype was evaluated. The accumulation of fat in mutant worms through Sudan black staining was observed under light microscope. While normal control worms showed the typical low fat content (FIG. 9A, I), extensive buildup of fat deposits in the intestines of mutant worms was found. Although fat accumulation was seen in young larvae, the buildup of fat was particularly pronounced in the L4 and adult stages (FIG. 9A, II, III). This finding suggests that the effect of the klf-3 deletion on fat content is incremental with the growth of mutant worms to adulthood. Fat deposition in the form of big lipid droplets under electron microscope was also observed. Normal worms had relatively small and few lipid droplets in their intestinal walls (FIG. 9B, panel I) whereas the klf-3 (ok1975) mutant worms displayed extensive accumulation of large lipid droplets in both the hypodermis and the intestinal wall (FIG. 9B, II, III). Thus, ablating the function of klf-3 gene results in severe reproductive defects and extensive fat deposition.

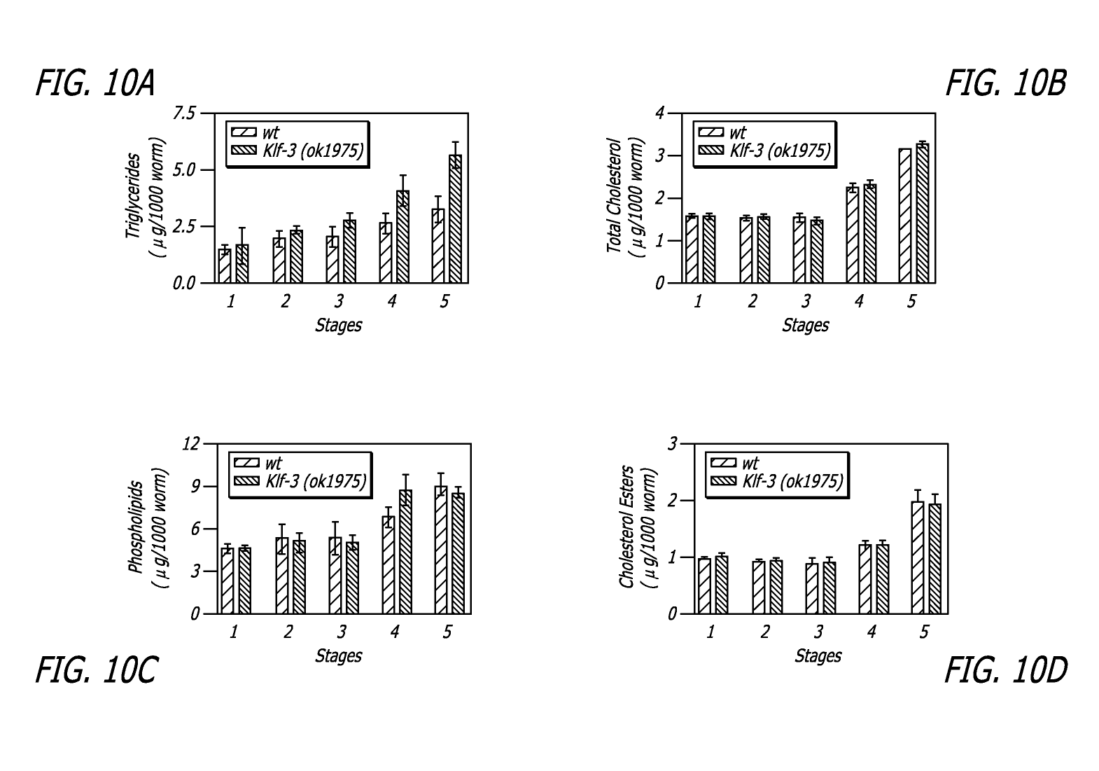

The total lipid content comprising triglycerides, phospholipids, and cholesterols was measured in the synchronized larval and adult stage of klf-3 mutant and compared to the same developmental stages of the wild type worm. (FIG. 10A-D) A significantly higher level of triglycerides was seen in most larval stages and adult mutant worms compared to same developmental stages of WT worms (FIG. 10A). The level of triglycerides increased with each developmental stage and was highest in the adult worms. While there was no difference in total cholesterol (FIG. 10B), phospholipids (FIG. 10C) and cholesterol esters (FIG. 10D) between wild type and mutant worm.

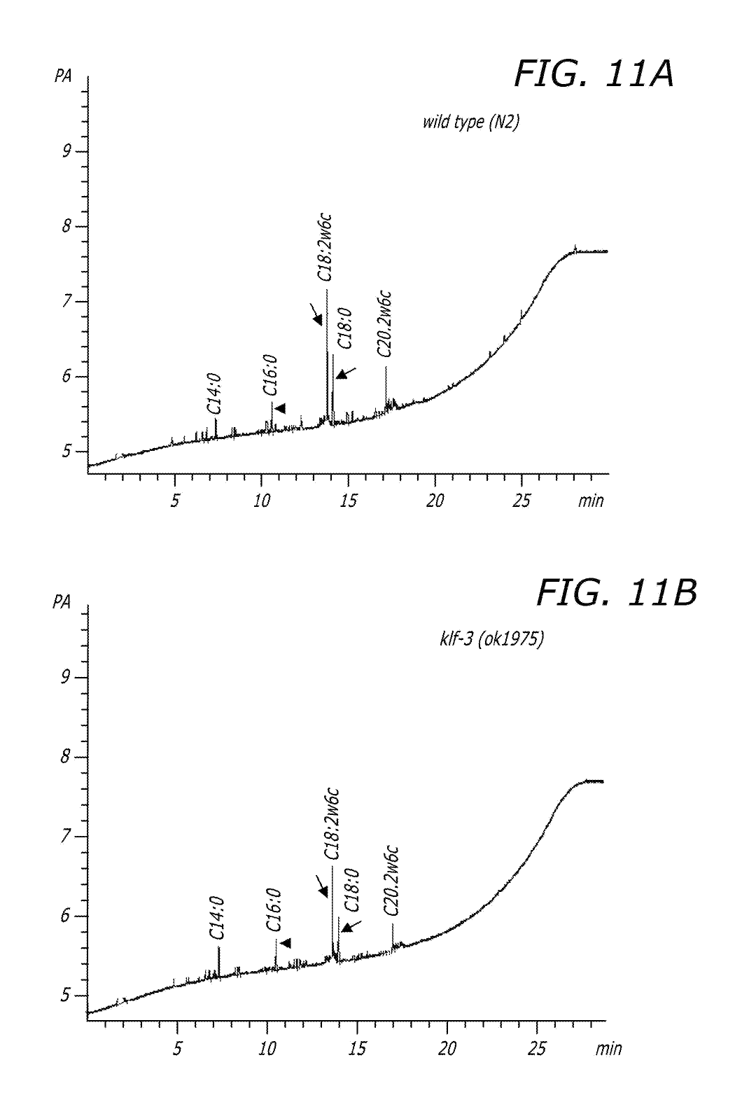

Fatty acid composition is altered in klf-3 (ok1975) mutants. The klf-3 (ok1975) mutant accumulates a large amount of fat in its intestine. It was anticipated that the accumulation of abnormally high fat contents resulted from the alteration of fatty acid (FA) composition in mutant worms. GC analysis of the total lipids to measure the FA composition of both mutant klf-3 (ok1975) and wild type (N2) worms grown under standard culture conditions and feeding on E. coli OP50 bacteria was used. The analysis revealed that an alteration in the long chain fatty acid composition had occurred in the mutant worms along with a substantial decrease in stearic acid (C18:0) and linoleic acid (C18:2w6c) (FIG. 11). A slight reduction in an unusual fatty acid, C20:2w6c, in the mutant worms was also noticed (FIG. 11). In addition, a reduction in palmitic acid (C16:0) was noticed in the mutant when compared to the wild type worm. In C. elegans, the pathway for unsaturated fatty acid synthesis begins with C16:0, which can be elongated to C18:0. Then C18:0 is subjected to desaturation to oleic acid (18:1 Δ9) and further desaturation and elongation of oleic acid results in the formation of polyunsaturated fatty acids (PUFAs). The results presented here indicate that the changes in C:18:0 and C18:2w6c or C20:2w6c in the klf-3 mutant worms influence desaturation and elongation and may have affected the FA metabolism pathway in its entirety. Furthermore, alterations in FA composition were associated with fat accumulation and other reproductive defects in the mutant worms, indicating the critical role of individual FAs in the physiological performance of an organism.

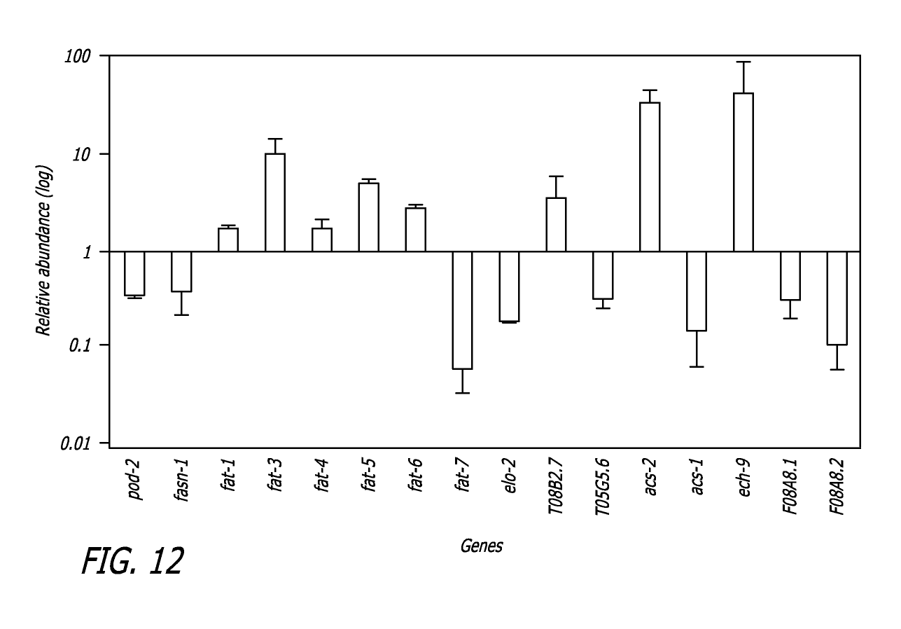

Klf-3 regulates genes involved in fatty acid metabolism pathway. The fat phenotype of klf-3 (ok1975) mutants suggests that klf-3 plays a key role in fat regulation and that its deletion may interfere with fatty acid synthesis, composition or metabolism related signal transduction. To test this hypothesis, qRT-PCR was used to assess the expression of a panel of genes involved in lipid metabolism pathways in klf-3 (ok1975) mutants and compared their expression to wild type worms. It was found that a substantial deletion in the klf-3 coding sequence produced a dramatic effect on multiple genes involved in the fatty acid β-oxidation (mitochondrial β-oxidation and peroxisomal β-oxidation) pathway. In addition, a mutation in klf-3 also resulted in dramatic changes in essential genes involved in fatty acid desaturation metabolic pathways (FIG. 12). Upon detailed examination of the RT-PCR data it was observed that the seven known genes predicted to function in mitochondria or peroxisomal β-oxidation pathways (acs-1, acs-2, ech-6 (T05G5.6), ech-9, F08A8.1, F08A8.2 and T08B2.7), altered expression in klf-3 (ok1975) mutants. Fatty acids in the form of Acyl-CoA molecules are broken down in mitochondria and/or peroxisomes to generate Acetyl-CoA. The observed alteration in the expression of the genes that facilitate this breakdown could interrupt the process of β-oxidation, ultimately leading to the accumulation of fat in klf-3 mutants.

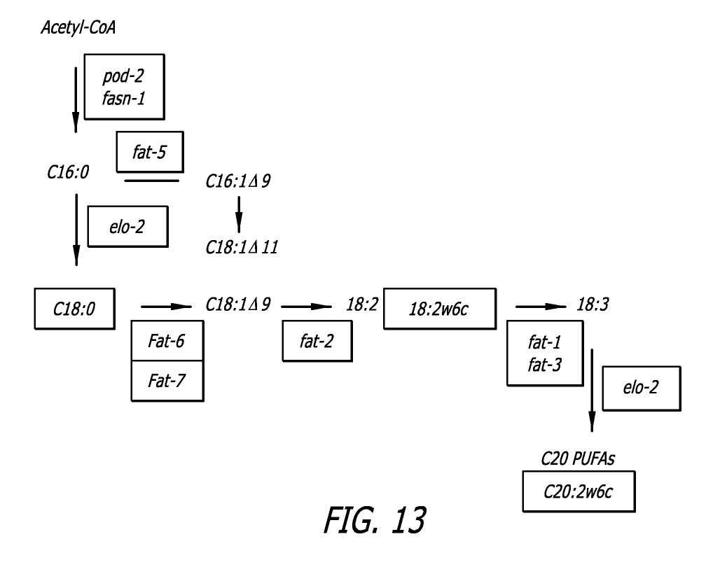

Through further analysis increased expression of seven fatty acid desaturases (fat-1, fat-3, fat-4, fat-5, and fat-6) (FIG. 12) and decreased expression of desaturate, fat-7, and elongase, elo-2, in the mutant worm was identified. The C. elegans fat-5, fat-6, and fat-7 genes encode A9-desaturases that catalyze the biosynthesis of monounsaturated C16:1 and C18:1 fatty acids from saturated C16:0 (palmitic acid) and C18:0 (stearic acid) fatty acids. The fat-5 gene encodes a palmitoyl-CoA desaturate, which specifically acts on palmitic acid (C16:0), while fat-6 and fat-7 genes encode stearoyl-CoA desaturases (SCD), which preferentially desaturate stearic acid (C18:0) (FIG. 13). The fat-3 gene encodes a Δ6-desaturases and is required for synthesis of C20 fatty acids. In klf-3 (ok1975) worms there was a significant increase in fat-3 expression (FIG. 12) and, conversely, a substantial decrease in the amount of C20:w6c (FIG. 11). With the exception of fat-2, the altered expression of the fat genes in the RT-PCR screen is consistent with data from lipid analysis which indicates a change in C18 and C20 fatty acid composition in klf-3 mutant worms (FIG. 11). Conceivably, increased expression of enzymes will increase consumption of their substrates, possibly leading to the formation of unsaturated fatty acids. Deletions in the klf-3 gene also affected two important enzymes, acetyl CoA carboxylase (ACC; pod-2) and fatty acid synthase (FAS; fasn-1) which are involved in fatty acid synthesis pathways (FIGS. 12 and 13). Acetyl-CoA carboxylase catalyses the irreversible carboxylation of acetyl-CoA to produce malonyl-CoA, while FAS catalyzes a series of multi-step chemical reactions through which FAS uses one acetyl-coenzyme A (CoA) and seven malonyl-CoA molecules to synthesize a 16-carbon palmitic acid. Overall, these results indicate the involvement of klf-3 in the control of fatty acid metabolism pathways. Klf-3 selectively acts on β-oxidation and on fatty acid desaturation pathway components to regulate their activity and integrate their crosstalk into a fat metabolism network.

Excess fat accumulation in klf-3 animals is likely to be the defect in mobilization of fat from the intestine after eating. To examine if the excess fat accumulation in klf-3 (ok1975) mutant animals is a result of excess food intake, food intake by the klf-3 mutant animals was quantified and compared with wild type (N2 strain), animals. Eat-2 (ad465) mutant animals which can not feed well were included as negative controls. The fluorescent intensity in the intestine of animals fed with their regular bacterial food, E. coli OP50 incorporated with BODIPY dye (Molecular Probes, #D-3822) was measured. The ingested BODIPY dye accumulates in the worm and its fluorescence can be measured. The BODIPY staining was performed as follows: C1-BODIPY-C12 was dissolved in DMSO, and a 5 mM stock solution was stored at −20 C. When needed, the stock solution was diluted in 1×PBS to 1 μM. Then 0.5 ml of the freshly prepared solution was applied to the surface of NGM (60×15 mm) plates seeded with E. coli OP50. The plates were allowed to air dry. The synchronized larval stages and adult hermaphrodites of wild type, eat-2 and klf-3 mutant animals were separately transferred onto NGM plates containing BODIPY E. coli. After 20-25 minutes of feeding, worms were washed in M9 buffer three times, transferred to NGM plates without bacteria. After 30 min of starvation, the worms were observed with a Zeiss Axioplan 2 imaging microscope. Although klf-3 mutant animals feed as WT, they accumulate more fat than WT suggesting that excess fat accumulation in klf-3 animals is likely to be the result of defect in mobilization of fat from the intestine after eating.

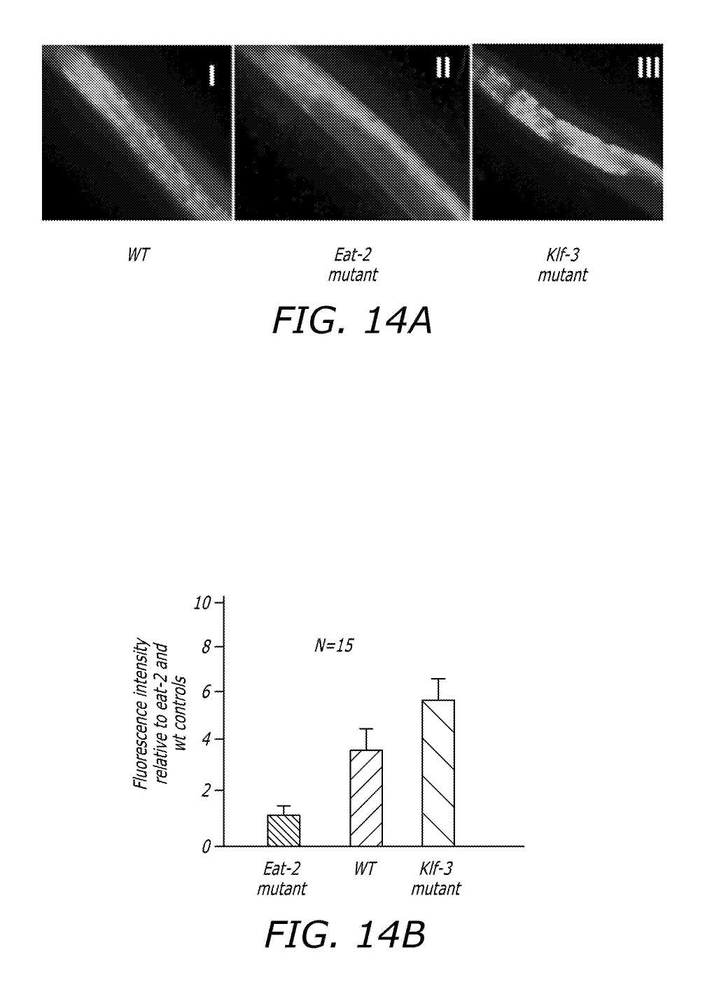

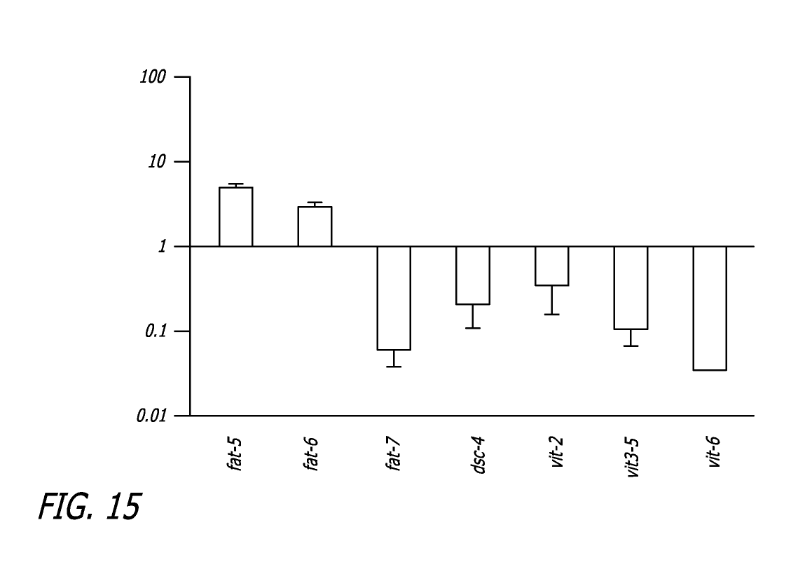

Klf-3 deletion affects the expression levels of genes related to mammalian lipoprotein assembly and transport. The profound effect of klf-3 (ok1975) mutation on the accumulation of fat in the form of triglycerides (FIG. 14) suggested that klf-3 plays a key role in the assembly and secretion of lipoproteins or lipoprotein like particles in the worm. Thus klf-3 functions to limit fat storage and helps in its mobilization to other tissues. In its absence, fat accumulation occurs in the intestine. Apolipoprotein B (apoB) and microsomal triglyceride transfer protein (MTP) are necessary for lipoprotein assembly. The lipid transfer activity of MTP is essential for the assembly and secretion of apoB-containing lipoproteins. The inhibition of MTP decreases lipoprotein secretion and lead to increased accumulation of lipids in the liver. To test this hypothesis, the C. elegans orthologues of mammalian MTP, Ce-dsc-4 and C. elegans vit genes, yolk protein and apolipoprotein B (apoB) were used as candidate targets of klf-3 to assess their expression. qRT-PCR was used to measure the mRNA levels of Ce-dsc-4 and C. elegans vit genes in klf-3 (ok1975) mutant. The expression of dsc-4 (5 fold), vit-2 (3 fold), vit-3, vit-4, vit-5 (10 fold) and vit-6 (15 fold) were reduced in klf-3 mutants (FIG. 15) suggesting that klf-3 may regulate the C. elegans orthologue of mammalian apoB and MTP in a similar process of lipid assembly and transport in C. elegans.

Discussion

The described data provides a detailed characterization of the worm klf-3 gene whose molecular properties and biological functions have not been understood prior to the described studies. It was shown that klf-3, together with klf-1 and klf-2, form a small gene family that falls into the superfamily of Krüppel-like transcription factors which is highly conserved and broadly expressed across metazoan lineages including humans. These KLFs bind to CACCC elements and GC-rich regions of DNA and mediate the activation or repression of transcription, performing diverse roles in proliferation, differentiation, and development. The klf-3 protein, like its two cousins, klf-1 and klf-2, contains a high amino acid sequence identity in the C-terminal DNA binding C₂H₂ zinc finger domains typical of all KLF members. The described genetic and phenotypic analyses of both WT and mutant klf-3 demonstrate that klf-3 acts as a negative regulator and plays an essential role in the fat metabolic network of C. elegans.

Fat storage has a pivotal role in the natural selection and evolution of metazoans. It offsets food shortage, a constant threat to animal survival and is most likely to have arisen first in the gut, the most ancient organ. The intestine-specificity of klf-3 and its identification as a key factor in fat regulation reinforces the early origin and adaptation of this genetic mechanism in lipid metabolism and energy homeostasis. This is corroborated by the presence and function of its cousin klf-1 in the intestine, although klf-1 is more widely expressed with roles in fat metabolism as well as in other cellular processes in C. elegans. Given the still incompletely defined and multi-factorial nature of fat storage, it is not surprising that both klf-3 and klf-1 have escaped RNAi screenings of fat regulation factors. Accordingly, klf-3 has been identified as the first of the KLF members now known to directly result in excessive fat deposition and big fat-droplet formation upon genetic mutation. The results support the stipulation that klf-3 has a significant role in modulating the activity of key metabolic and signaling pathways, which collectively manifest in a negative regulatory mechanism for fat storage and lipid metabolism.

Regulation of fat storage involves a complex array of signaling pathways which govern food intake, nutrient transport, and metabolite flow in a highly concerted manner in both worms and humans. In worms as in mammals, SCDs play key roles in keeping a proper level and ratio of saturated to unsaturated fatty acids, acting in the intestine and under the strict control of the transcription factor NHR-80. A fat-5/fat-6/fat-7 triple mutant produces sterile adults with imbalanced fatty acid composition, reduced movement, and early death, while an NHR-80 mutation down-regulates all of them. Although desaturases have no linkage to the lifespans of worms or mammals, a change in the ratio of saturated to monounsaturated fatty acids contributes to shortened lifespan in worms. It was found that in spite of greatly reduced fertility in klf-3 mutant worms, they lived nearly as long as wild type worms.

The described quantitative RT-PCR analysis showed that the expression of genes devoted to the regulation of fatty acid desaturation and fatty acid β-oxidation pathways were changed in klf-3 (ok1975) mutants. Substantial increases in fat-1, fat-3, fat-4, fat-5, and fat-6 expression, but significant decrease in the expression of fat-7, were observed suggesting klf-3 could differentially regulate these fat genes in the maintenance of proper enzymatic activities via balancing actions. Klf-3 maintains the balance of saturated and monounsaturated fatty acids by regulating the expression of fatty acid A9-desaturase genes, fat-1, fat-3, fat-4, fat-5, fat 6 and fat-7, which in turn catalyze the biosynthesis of monounsaturated C16:1 and C18:1 fatty acids from saturated C16:0 and C18:0 fatty acids. An imbalance in fatty acid saturation has been linked to numerous pathological conditions. Thus, it is possible that the observed sterile or semi-sterile phenotype in klf-3 (ok1975) worms results from the improper ratio of saturated and monounsaturated fats. In support of above hypothesis, the lipid analysis data indicates an alteration in the relative abundance of C16:0, C18:0, and C18:2w6c in klf-3 (ok1975) mutants. Deletion in the klf-3 gene also results in a several fold increase in fat-3 expression. As fat-3 is required for C20 synthesis a change in the amount of C20 fatty acids was anticipated. In fact, a reduction in C20:2w6c fatty acids in the klf-3 mutant worms was observed. This suggests that a change in C20 occurs because the elevated expression of fat-3 results in a noticeable effect on C20 synthesis easily detectable in total fatty acids from GC analysis. The analysis thus far suggests that klf-3 is involved in the breakdown of fatty acids by affecting the genes hypothesized to participate in the β-oxidation pathway. A mutation in nhr-49 (nr2041) results in increased fat due to the reduced expression of β-oxidation genes. Klf-3 may manage over all fat storage by a similar mechanism as nhr-49. Consequently, klf-3 selectively acts on key signaling modules to mediate pathway activities and integrate their crosstalk into a fat regulation network.

The data suggests a link between fat accumulation and reproductive deficiency given the observation that the disruption of fat regulation in klf-3 mutants contributes to defects in germ cell differentiation and oocyte development. This deregulation is particularly interesting, considering that germ cells proliferate and develop during early larval development in C. elegans. Although klf-3 is found in neither germ cells nor oocytes, its transcript is constantly and highly expressed during larval development, suggesting that its function and regulation in larvae is required for later stage-specific cell proliferation. In support of this, young larvae carrying the klf-3 deletion displayed grossly normal phenotypes, whereas L4 or young adult mutant worms manifested morphological and functional abnormalities in multiple and varied ways. Most likely, both the extensive fat deposition and severe reproductive sterility associated with klf-3 mutants results from damage that takes place in the course of larval development and gradually accumulates. Reproductive defects could be secondary to the buildup of fat storage, given that the loss-of function of klf-3 would primarily affect the functions of the intestine. The intestines of worms are major endocrine systems and tissues engaged in nutrient sensing and energy metabolism positioned close to sexual organs. Upon klf-3 mutation, increasing fat deposition could compromise these functions, ultimately exerting a negative impact on reproduction.

C. elegans klf-3 has a role in the regulation of FA β-oxidation and germline development. This finding is significant because it may reveal a KLF-3 control mechanism in nutrition sensitive cell proliferation and provides a new link between germline development and lipid metabolism. Understanding this link is important because it may provide a clue to understanding obesity and fertility defect in human. The KLF-mediated regulatory networks that govern adipogenesis are complex and still poorly understood and, in particular, their involvement in FA β-oxidation and/or germline proliferation is not known. Given that FA β-oxidation regulation is central to lipid metabolism and energy homeostasis, its perturbation and dysfunction lead to common disorders such as diabetes, obesity, atherosclerosis, and accelerated aging. There are more than 20 known inherited diseases of FA β-oxidation pathway. On the other hand reproduction is an energy-concentrated process, which is modulated by the availability of nutrients including lipids and by the status of their metabolism. During larval development the worm make an assessment of nutritional sufficiency which influences germline proliferation. During C. elegans germline development the undifferentiated germ cells proliferate, forming a progenitor pool that maintains gamete production throughout reproductive adulthood. Thus the C. elegans germline is a classical system in which signaling promotes the undifferentiated versus differentiated fate. The molecular genetic analysis in the worm can lead to the discovery of previously unsuspected human genes that may play a key role in FA β-oxidation and germline proliferation. Such genes will define new candidates for the underlying cause of metabolic disease and may serve novel targets for new therapies.

Moreover, the profound effect of klf-3 mutation on the accumulation of triglycerides suggests that klf-3 functions to limit fat storage and plays a role in its mobilization to other tissues and mutation in klf-3 reduce lipid absorption and mobilization leading to fat accumulation in the intestine. The accumulation of high cholesterol and lipids are linked to a number of interrelated pathological conditions and diseases, including obesity, type II diabetes, and fatty liver. This set of conditions commonly known as metabolic disorders are affecting a rapidly increasing number of individuals. Treatments for diseases associated with metabolic syndrome have focused primarily on individual elements, such as high LDL-cholesterol (targeted by the cholesterol-lowering statin drugs). Statins enhance lipoprotein catabolism and reduce plasma cholesterol. Despite success of statins, a significant numbers of statin-treated patients developed adverse coronary events. Therefore, more effective drugs that can be used alone or in combination with statins to treat the components of metabolic disorder are needed. One attractive approach might be to target the genetic switches that promote lipid synthesis.

The KLF family plays vital transcriptional roles in diverse cellular processes in both mice and humans. Several KLFs are implicated in association with diabetes due to their residence activities in adipose tissue, pancreas, liver, or muscle; furthermore they regulate adipocyte differentiation, promote lipogenesis, or tune glucose/lipid homeostasis. When compared to their mammalian counterparts, the observations made concerning the expression and actions of worm klf-3 provide insight into the regulation of fat storage and adiposity in several ways. First, they link KLF to an essential negative regulatory mechanism of fat storage in the intestine as the major site of early origin. This makes the worm intestine a useful model in future studies to address the positive and negative impact of neuroendocrine signals on lipogenesis and fat deposition as in mammals. Second, they connect klf-3 expression to larval development and reveal the significant pathogenic effects of fat storage on germ cell function and worm reproduction. This causal link presents an example when looking into parallel conditions in humans, namely, obesity-associated reproductive deficiency and gestational diabetes. Third, they relate the regulatory function of klf-3 to the desaturases and n-oxidation signaling pathway that control fatty acid metabolism. Given the universal nature of these three genetic modules in animals, the newly uncovered aspects of fat regulation may identify conserved organismal features and direct research avenues to therapeutic applications.

### Example 3

### 3T3-L1 Pre-Adipocyte Cell Transfection Studies

Ce-klf-3 cloning. Full-length cDNA of Ce-klf-3 was amplified by PCR using primer pair: 5′-TCAAGCTTATGCTGAAAATGGAACAAAG-3′ (SEQ ID NO:107) and 5′-CAGGATCCATTGTGCTATGGCGCTTC-3′ (SEQ ID NO:108) from the cDNA library. It was first cloned in TOPO vector. Then the cDNA was digested with BamHI at the 5′ and Hind III at the 3′ of the sequence and ligated into mammalian cells expression vector (pEGFP-N1, BD Biosciences Clontech) containing gfp reporter gene.

Cell culture, induction of adipocyte differentiation, and transfection. 3T3-L1 pre-adipocyte cells were cultured in high-glucose Dulbecco's modified Eagle's medium (HG-DMEM) with 10% (v/v) heat inactivated fetal bovine serum (FBS) at 37° C. and 5% CO₂. The cells were plated in a six-well plate. Next day the cells became confluent. Five groups of cells were set up for this study: (1) negative group one: the cells were cultured in standard medium; (2) negative group two: the cells were cultured in standard medium but transfected with ce-klf-3 using FuGENE® 6 Transfection Reagent (Roche Applied Science) on day 0, day 2 and day 4 according to manufacturer's recommendations; (3) positive group one: the 3T3-L1 cells were induced (designated day 0) by replacing the medium with differentiation medium (HG-DMEM, 10% FBS, 2 μg/ml insulin, 1 μM dexamethasone and 0.5 mM 3-isobutyl-1-methylxanthine). On day 2, cells were post-induced by changing to second medium (HG-DMEM, 10% FBS, 2 μg/ml insulin). On day 4 the cells were maintained in standard medium and on day 5 cells were visualized for intracellular lipid droplets staining or harvested for further studies; (4) positive group two: the cells were induced as above and transfected with pEGFP-N1 vector alone on day 0, day 2 and day; (5) experimental group: the cells were induced as above and transfected with Ce-klf-3 on day 0, day 2, day 4. Approximately, 2.5 μg of plasmid DNA was used for each transfection in a volume of 6 μl at 2:1 ratio to FuGENE® 6 Transfection Reagent.

Fat staining of the induced/transfected cells. The differentiated cells were visualized for intracellular lipid droplets by Oil Red O (Sigma) staining on day 5. Cells were fixed in 2% formaldehyde for 1 hour at room temperature. Cells were then washed quickly in PBS for 2 minutes three times. Oil Red O (0.5% in isopropanol) was diluted with water (3:2), filtered and used for the staining. The cells were stained for 1 hour at room temperature, rinsed three times with PBS for 2 minutes each time. The intracellular lipid droplets were visualized as red droplets under the light-microscope.

The results of this study demonstrate that over-expression of worm klf-3 in mouse 3T3-L1 preadipocyte cells significantly suppress the cell differentiation processes.

### Example 4

### Rescue of the Klf-3 Mutant by the Mammalian KLFs

Currently, the human KLF family consists of 17 members. The fat phenotype of klf-3 mutants are rescued by mammalian klf-2, klf-3, klf-4, klf-5, klf-6, klf-7, or klf-15. It will also be possible to include other members of mammalian KLF in rescue experiments.

In all seven rescue constructs are made. Each rescue construct contains the cDNA of an individual mammalian klf gene fused to gfp and is controlled by the C. elegans klf-3 promoter to ensure that the expression of the mammalian genes is in the intestine at the correct time throughout C. elegans development. The rescue experiment is performed and the transgenes are assayed for fat accumulation, germline development, and reproduction.

All constructs are translational fusion constructs in which the klf-3 promoter and the cDNA or full coding sequences including introns of the test genes are PCR amplified using C. elegans genomic DNA as a template. Then cDNA or full coding sequences are cloned between the klf-3 promoter and the gfp reporter into vector pPD95.75. For negative controls, appropriate constructs are designed in which the coding sequence of test genes have been replaced by non-specific C. elegans genes that are not involved in fat metabolism, such as C. elegans STIP. The plasmid are prepared using the CONCERT™ rapid plasmid miniprep system (Invitrogen), and then injected into the gonadal syntium of individual adult klf-3 mutant hermaphrodites. The procedure for microinjection into the mutant worm gonad is necessarily the same as injecting into gonad of wild type worm. The pRF4 plasmid, which contains the dominant marker rol-6 (su1006) encoding a mutant collagen, is co-injected (50-80 ng/μl) to confer a visible roller phenotype to transgenic worms. The F2 roller animals are observed for gfp expression. The transgenes are analyzed for reproduction and subject to Sudan black staining for fat accumulation.

Additionally, the klf-3 mutant is rescued by genetic crosses. For this wild type C. elegans transgenes are created expressing the individual rescue constructs and then introduced this into klf-3 mutant worm by genetic crosses.

Unless otherwise indicated, all numbers expressing quantities of ingredients, properties such as molecular weight, reaction conditions, and so forth are to be understood as being modified in all instances by the term “about.” Accordingly, unless indicated to the contrary, the numerical parameters set forth are approximations that may vary depending upon the desired properties sought to be obtained. At the very least, each numerical parameter should at least be construed in light of the number of reported significant digits and by applying ordinary rounding techniques. Notwithstanding that the numerical ranges and parameters are approximations, the numerical values set forth in the specific examples are reported as precisely as possible. Any numerical value, however, inherently contains certain errors necessarily resulting from the standard deviation found in their respective testing measurements.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

While certain embodiments according to this invention are described herein, variations of those embodiments will become apparent to those of ordinary skill in the art upon reading the foregoing description.

Furthermore, numerous references have been made to patents and printed publications throughout this specification. Each of the above cited references and printed publications are herein individually incorporated by reference in their entirety.

What is claimed is:

1. A method of regulating fat accumulation comprising upregulating or downregulating the activity of at least one Krüppel-like factor (KLF).

2. The method of claim 1 wherein said upregulating or downregulating the activity of at least one KLF comprises administering an agent that potentiates or inhibits the expression of KLF genes.

3. The method of claim 1 wherein said upregulating or downregulating the activity of at least one KLF comprises administering an agent that potentiates or inhibits the activity of KLF proteins.

4. The method according to claim 1 wherein said KLF is a human KLF selected from the group consisting of klf-1, klf-2, klf-3, klf-4, klf-5, klf-6, klf-7, klf-8, klf-9, klf-10, klf-11, klf-12, klf-13, klf-14, klf-15, klf-16 and klf-17.

5. The method according to claim 1 wherein said KLF is a Caenorhabditis elegans KLF selected from the group consisting of klf-1, klf-2, and klf-3.

6. A method according to claim 2 wherein said agent is selected from the group consisting of DNA, RNA, cDNA, siRNA, and shRNA.

7. The method according to claim 3 wherein said agent is selected from the group consisting of proteins, monoclonal antibodies, polyclonal antibodies, peptides, and small molecules.

8. A method of suppressing or stimulating cell differentiation processes comprising upregulating or downregulating the activity of at least one KLF.

9. The method according to claim 8 wherein said KLF is a human KLF selected from the group consisting of klf-1, klf-2, klf-3, klf-4, klf-5, klf-6, klf-7, klf-8, klf-9, klf-10, klf-11, klf-12, klf-13, klf-14, klf-15, klf-16 and klf-17.

10. The method according to claim 8 wherein said KLF is a C. elegans KLF selected from the group consisting of klf-1, klf-2, and klf-3.

11. A method according to claim 8 wherein said upregulating comprises administering an agent that mimics or stimulates KLF activity.

12. A method according to claim 11 wherein said agent is selected from the group consisting of DNA, RNA, cDNA, siRNA, shRNA, protein, monoclonal antibodies, polyclonal antibodies, peptides or small molecules.

13. A method according to claim 8 wherein said upregulating comprises stimulating KLF gene activity.

14. A method according to claim 8 wherein said downregulating comprises mutating genes encoding said KLF genes that are expressed by activity of said KLFz.

15. A method according to claim 8 wherein said downregulating comprises administering an agent that blocks a biological site required for the activity of said KLFs or otherwise inhibits the activity of said KLFs.

16. A method according to claim 15 wherein said agent is a klf-1 antagonist, a klf-3 antagonist, DNA, RNA, cDNA, protein, a monoclonal antibody, a polyclonal antibody or a peptide.

17. A cell line transfected with at least one Krüppel-like factor (KLF).

18. The cell line according to claim 17 wherein said KLF is a human KLF selected from the group consisting of klf-1, klf-2, klf-3, klf-4, klf-5, klf-6, klf-7, klf-8, klf-9, klf-10, klf-11, klf-12, klf-13, klf-14, klf-15, klf-16 and klf-17.

19. The method according to claim 17 wherein said KLF is a C. elegans KLF selected from the group consisting of klf-1, klf-2, and klf-3.

20. A cell line according to claim 19 wherein said KLF is klf-1 or klf-3.


SEQUENCE LISTING (110 sequences)

SEQ ID NO: 1 - protein, 497 aa, Caenorhabditis elegans
MTSSSINATSSREPLIHIPNKPSLSIDPLARADRSVHLSPSFFQPGLSSISSENDDAEQRIHEATRSFNQFGHQFYESIRELSESTSAYERLKANALRRKLENTFGNDSGAATSSSSSSVFERQSDFSAFAPYKNSHFDSTQSLFQPTTSFNDRLEQIKSDFIAEHQKSMSSFSGFNTPTTALGAAFQGMQVKKSAFAPVLLRENLDTRRPGGHLISDILNRDPLPQSRNLNLNMARNVPIRLIHSTSNFDIASSSSGDSGHQDHESIVVEDADMDSPTSPCVKRSAMNFDLRDEPLTVNVESVSSTSDLPSSVSSSVNSFVYQNFDPLEFKRKIDELTASACLAVMPDANGQVDPMAIKTQLDAIKKQMEEHQTHMAEASQRLHVDSSLEDSNCEPSPSSSYDASEPSVKRLHHCTHPNCGKVYTKSSHLKAHFRTHTGEKPYECSWDGCDWRFARSDELTRHYRKHTGDRPFKCSQCSRAFSRSDHLSLHMKRHF

SEQ ID NO: 2 - protein, 354 aa, Caenorhabditis elegans
MTTIYTCSSFLYFLFLGGKKSLLKFSVCYHRSDRSGVTTPLFWSLGSSLFCRPEEMYSSIVPSSSNGYYHQSQYQNAHHQHHQQHYHQQSHHHYNGAAAAPVINVHNYHFHTGPVNNQVIEQHYNHHNHVQQTFEENIPQGPHSSFNFSHDFPQQNNSETHLNHMEPSMTPMEHQNSGSSTPYPFEAKLFVPSPAASVSSYSFSSDLSGKDEEDPRIPLKDRGRVYHPQSTEKPKKVPSKRRDKATLDRLRVHKCFYQGCGKVYTKSSHLTAHERVHSGEKPYPCEWPGCSWRFARSDELTRHYRKHTGAKPFACKECSRKFSRSDHLQLHMKRHETDEQDDGMDDFKDFMTFI

SEQ ID NO: 3 - protein, 705 aa, Caenorhabditis elegans
MSNEEIESAYTNTIEQILELSGNTAAAYIPILGGKSIELAISTMEEYLVMLKNLKEEYEKEPTHLSSVSPRANDSMLLRRIQLAHTSKSRPTLQRTSPEVARFIGSPMEIDNLGISRSDTIAEKSSFLDPTMMMDKTTHERGFLADVSDMNGTVRSRQASFHFNYTSPVPAEKTRRSSGIQHANSTIDFDADVSSFFDSTQIHRRSRQQAGREPSIALINDGLRTPITLNNTILLSDSETTPTNLQTLWNVKLRPGSNRKIDNEIRKEIQRQLACDPQFLTMLKLLLPLLIYPVPFFAQNLVIDVSRNAIQEYVSSMSTHFKDEILKAKLPPMDASRFICGVTLKDTTVTDFNFSKFSSELAGPYKDYYLNITDFSFTISGSYVRNFLFFKFYDTFSVKIKFKNVSLGALVPTFANGYPFLHGVINCGFSPETVEDPVTNGWFVTSLIREAVEENIKEAICHILVQLLDSKIRSDIHELQVDFPIFSNTSEMSFRMFKKAPYTSSTKQIRYFLEGEIEYPASEPAVFLPNEVSDEIPKRHVAYHIHEKLLAEMLEAMCEKGLFDGNFTSLPSMKPVSYLCLSASAKIQGISNTITIIIHITSLETYPDDQEKSQLRSFMVTPLHNSEHELFIRLKSQLANRWLDNTFTEQLFNHLSVVLSDNFRLPLPFIHNSSISDVIFLPNADYFTILSNFDFHQNDSQFRNR

SEQ ID NO: 4 - protein, 362 aa, Homo sapiens [sequence omitted]

SEQ ID NO: 5 - protein, 355 aa, Homo sapiens
MALSEPILPSFSTFASPCRERGLQERWPRAEPESGGTDDDLNSVLDFILSMGLDGLGAEAAPEPPPPPPPPAFYYPEPGAPPPYSAPAGGLVSELLRPELDAPLGPALHGRFLLAPPGRLVKAEPPEADGGGGYGCAPGLTRGPRGLKREGAPGPAASCMRGPGGRPPPPPDTPPLSPDGPARLPAPGPRASFPPPFGGPGFGAPGPGLHYAPPAPPAFGLFDDAAAAAAALGLAPPAARGLLTPPASPLELLEAKPKRGRRSWPRKRTATHTCSYAGCGKTYTKSSHLKAHLRTHTGEKPYHCNWDGCGWKFARSDELTRHYRKHTGHRPFQCHLCDRAFSRSDHLALHMKRHM

SEQ ID NO: 6 - protein, 345 aa, Homo sapiens
MLMFDPVPVKQEAMDPVSVSYPSNYMESMKPNKYGVIYSTPLPEKFFQTPEGLSHGIQMEPVDLTVNKRSSPPSAGNSPSSLKFPSSHRRASPGLSMPSSSPPIKKYSPPSPGVQPFGVPLSMPPVMAAALSRHGIRSPGILPVIQPVVVQPVPFMYTSHLQQPLMVSLSEEMENSSSSMQVPVIESYEKPISQKKIKIEPGIEPQRTDYYPEEMSPPLMNSVSPPQALLQENHPSVIVQPGKRPLPVESPDTQRKRRIHRCDYDGCNKVYTKSSHLKAHRRTHTGEKPYKCTWEGCTWKFARSDELTRHFRKHTGIKPFQCPDCDRSFSRSDHLALHRKRHMLV

SEQ ID NO: 7 - protein, 470 aa, Homo sapiens
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 8 - protein, 457 aa, Homo sapiens
MATRVLSMSARLGPVPQPPAPQDEPVFAQLKPVLGAANPARDAALFPGEELKHAHHRPQAQPAPAQAPQPAQPPATGPRLPPEDLVQTRCEMEKYLTPQLPPVPIIPEHKKYRRDSASVVDQFFTDTEGLPYSINMNVFLPDITHLRTGLYKSQRPCVTHIKTEPVAIFSHQSETTAPPPAPTQALPEFTSIFSSHQTAAPEVNNIFIKQELPTPDLHLSVPTQQGHLYQLLNTPDLDMPSSTNQTAAMDTLNVSMSAAMAGLNTHTSAVPQTAVKQFQGMPPCTYTMPSQFLPQQATYFPPSPPSSEPGSPDRQAEMLQNLTPPPSYAATIASKLAIHNPNLPTTLPVNSQNIQPVRYNRRSNPDLEKRRIHYCDYPGCTKVYTKSSHLKAHLRTHTGEKPYKCTWEGCDWRFARSDELTRHYRKHTGAKPFQCGVCNRSFSRSDHLALHMKRHQN

SEQ ID NO: 9 - protein, 283 aa, Homo sapiens
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGDKGNGDASPDGRRRVHRCHFNGCRKVYTKSSHLKAHQRTHTGEKPYRCSWEGCEWRFARSDELTRHFRKHTGAKPFKCSHCDRCFSRSDHLALHMKRHL

SEQ ID NO: 10 - protein, 241 aa, Homo sapiens
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGEKPYRCSWEGCEWRFARSDELTRHFRKHTGAKPFKCSHCDRCFSRSDHLALHMKRHL

SEQ ID NO: 11 - protein, 237 aa, Homo sapiens
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGDKGNGDASPDGRRRVHRCHFNGCRKVYTKSSHLKAHQRTHTGVFPGLTTWPCT

SEQ ID NO: 12 - protein, 302 aa, Homo sapiens
MDVLASYSIFQELQLVHDTGYFSALPSLEETWQQTCLELERYLQTEPRRISETFGEDLDCFLHASPPPCIEESFRRLDPLLLPVEAAICEKSSAVDILLSRDKLLSETCLSLQPASSSLDSYTAVNQAQLNAVTSLTPPSSPELSRHLVKTSQTLSAVDGTVTLKLVAKKAALSSVKVGGVATAAAAVTAAGAVKSGQSDSDQGGLGAEACPENKKRVHRCQFNGCRKVYTKSSHLKAHQRTHTGEKPYKCSWEGCEWRFARSDELTRHYRKHTGAKPFKCNHCDRCFSRSDHLALHMKRHI

SEQ ID NO: 13 - protein, 359 aa, Homo sapiens
MVDMDKLINNLEVQLNSEGGSMQVFKQVTASVRNRDPPEIEYRSNMTSPTLLDANPMENPALFNDIKIEPPEELLASDFSLPQVEPVDLSFHKPKAPLQPASMLQAPIRPPKPQSSPQTLVVSTSTSDMSTSANIPTVLTPGSVLTSSQSTGSQQILHVIHTIPSVSLPNKMGGLKTIPVVVQSLPMVYTTLPADGGPAAITVPLIGGDGKNAGSVKVDPTSMSPLEIPSDSEESTIESGSSALQSLQGLQQEPAAMAQMQGEESLDLKRRRIHQCDFAGCSKVYTKSSHLKAHRRIHTGEKPYKCTWDGCSWKFARSDELTRHFRKHTGIKPFRCTDCNRSFSRSDHLSLHRRRHDTM

SEQ ID NO: 14 - protein, 257 aa, Homo sapiens
MVDMDKLINNLEVQLNSEGGSMQVFKQVTASVRNRDPPEIEYRSNMTSPTLLDANPMENPALFNDIKIEPPEELLASDFSLPQVEPVDLSFHKPKAPLQPASMLQAPIRPPKPQSSPQTLVVSTSTSDMSTSANIPTVLTPGSVLTSSQSTGSQQILHVIHTIPSVSLPNKMGGLKTIPVVVQSLPMVYTTLPADGGPAAITVPLIGGDGKNAGSVKVDPTSMSPLEIPSDSEESTIESGSSALQSLQGLQQEREAL

SEQ ID NO: 15 - protein, 244 aa, Homo sapiens
MSAAAYMDFVAAQCLVSISNRAAVPEHGVAPDAERLRLPEREVTKEHGDPGDTWKDYCTLVTIAKSLLDLNKYRPIQTPSVCSDSLESPDEDMGSDSDVTTESGSSPSHSPEERQDPGSAPSPLSLLHPGVAAKGKHASEKRHKCPYSGCGKVYGKSSHLKAHYRVHTGERPFPCTWPDCLKKFSRSDELTRHYRTHTGEKQFRCPLCEKRFMRSDHLTKHARRHTEFHPSMIKRSKKALANAL

SEQ ID NO: 16 - protein, 480 aa, Homo sapiens
MLNFGASLQQTAEERMEMISERPKESMYSWNKTAEKSDFEAVEALMSMSCSWKSDFKKYVENRPVTPVSDLSEEENLLPGTPDFHTIPAFCLTPPYSPSDFEPSQVSNLMAPAPSTVHFKSLSDTAKPHIAAPFKEEEKSPVSAPKLPKAQATSVIRHTADAQLCNHQTCPMKAASILNYQNNSFRRRTHLNVEAARKNIPCAAVSPNRSKCERNTVADVDEKASAALYDFSVPSSETVICRSQPAPVSPQQKSVLVSPPAVSAGGVPPMPVICQMVPLPANNPVVTTVVPSTPPSQPPAVCPPVVFMGTQVPKGAVMFVVPQPVVQSSKPPVVSPNGTRLSPIAPAPGFSPSAAKVTPQIDSSRIRSHICSHPGCGKTYFKSSHLKAHTRTHTGEKPFSCSWKGCERRFARSDELSRHRRTHTGEKKFACPMCDRRFMRSDHLTKHARRHLSAKKLPNWQMEVSKLNDIALPPTPAPTQ

SEQ ID NO: 17 - protein, 469 aa, Homo sapiens
MEERMEMISERPKESMYSWNKTAEKSDFEAVEALMSMSCSWKSDFKKYVENRPVTPVSDLSEEENLLPGTPDFHTIPAFCLTPPYSPSDFEPSQVSNLMAPAPSTVHFKSLSDTAKPHIAAPFKEEEKSPVSAPKLPKAQATSVIRHTADAQLCNHQTCPMKAASILNYQNNSFRRRTHLNVEAARKNIPCAAVSPNRSKCERNTVADVDEKASAALYDFSVPSSETVICRSQPAPVSPQQKSVLVSPPAVSAGGVPPMPVICQMVPLPANNPVVTTVVPSTPPSQPPAVCPPVVFMGTQVPKGAVMFVVPQPVVQSSKPPVVSPNGTRLSPIAPAPGFSPSAAKVTPQIDSSRIRSHICSHPGCGKTYFKSSHLKAHTRTHTGEKPFSCSWKGCERRFARSDELSRHRRTHTGEKKFACPMCDRRFMRSDHLTKHARRHLSAKKLPNWQMEVSKLNDIALPPTPAPTQ

SEQ ID NO: 18 - protein, 512 aa, Homo sapiens
MHTPDFAGPDDARAVDIMDICESILERKRHDSERSTCSILEQTDMEAVEALVCMSSWGQRSQKGDLLRIRPLTPVSDSGDVTTTVHMDAATPELPKDFHSLSTLCITPPQSPDLVEPSTRTPVSPQVTDSKACTATDVLQSSAVVARALSGGAERGLLGLEPVPSSPCRAKGTSVIRHTGESPAACFPTIQTPDCRLSDSREGEEQLLGHFETLQDTHLTDSLLSTNLVSCQPCLHKSGGLLLTDKGQQAGWPGAVQTCSPKNYENDLPRKTTPLISVSVPAPPVLCQMIPVTGQSSMLPAFLKPPPQLSVGTVRPILAQAAPAPQPVFVGPAVPQGAVMLVLPQGALPPPAPCAANVMAAGNTKLLPLAPAPVFITSSQNCVPQVDFSRRRNYVCSFPGCRKTYFKSSHLKAHLRTHTGEKPFNCSWDGCDKKFARSDELSRHRRTHTGEKKFVCPVCDRRFMRSDHLTKHARRHMTTKKIPGWQAEVGKLNRIASAESPGSPLVSMPASA

SEQ ID NO: 19 - protein, 402 aa, Homo sapiens
MNIHMKRKTIKNINTFENRMLMLDGMPAVRVKTELLESEQGSPNVHNYPDMEAVPLLLNNVKGEPPEDSLSVDHFQTQTEPVDLSINKARTSPTAVSSSPVSMTASASSPSSTSTSSSSSSRLASSPTVITSVSSASSSSTVLTPGPLVASASGVGGQQFLHIIHPVPPSSPMNLQSNKLSHVHRIPVVVQSVPVVYTAVRSPGNVNNTIVVPLLEDGRGHGKAQMDPRGLSPRQSKSDSDDDDLPNVTLDSVNETGSTALSIARAVQEVHPSPVSRVRGNRMNNQKFPCSISPFSIESTRRQRRSESPDSRKRRIHRCDFEGCNKVYTKSSHLKAHRRTHTGEKPYKCTWEGCTWKFARSDELTRHYRKHTGVKPFKCADCDRSFSRSDHLALHRRRHMLV

SEQ ID NO: 20 - protein, 288 aa, Homo sapiens
MAAAAYVDHFAAECLVSMSSRAVVHGPREGPESRPEGAAVAATPTLPRVEERRDGKDSASLFVVARILADLNQQAPAPAPAERREGAAARKARTPCRLPPPAPEPTSPGAEGAAAAPPSPAWSEPEPEAGLEPEREPGPAGSGEPGLRQRVRRGRSRADLESPQRKHKCHYAGCEKVYGKSSHLKAHLRTHTGERPFACSWQDCNKKFARSDELARHYRTHTGEKKFSCPICEKRFMRSDHLTKHARRHANFHPGMLQRRGGGSRTGSLSDYSRSDASSPTISPASSP

SEQ ID NO: 21 - protein, 323 aa, Homo sapiens
MSAAVACLDYFAAECLVSMSAGAVVHRRPPDPEGAGGAAGSEVGAAHPESALPGPGPSGPASVPQLPQVPAPSPGAGGAAPHLLAASVWADLRGSSGEGSWENSGEAPRASSGFSDPIPCSVQTPCSELAPASGAAAVCAPESSSDAPAVPSAPAAPGAPAASGGFSGGALGAGPAPAADQAPRRRSVTPAAKRHQCPFPGCTKAYYKSSHLKSHQRTHTGERPFSCDWLDCDKKFTRSDELARHYRTHTGEKRFSCPLCPKQFSRSDHLTKHARRHPTYHPDMIEYRGRRRTPRIDPPLTSEVESSASGSGPGPAPSFTTCL

SEQ ID NO: 22 - protein, 416 aa, Homo sapiens
MVDHLLPVDENFSSPKCPVGYLGDRLVGRRAYHMLPSPVSEDDSDASSPCSCSSPDSQALCSCYGGGLGTESQDSILDFLLSQATLGSGGGSGSSIGASSGPVAWGPWRRAAAPVKGEHFCLPEFPLGDPDDVPRPFQPTLEEIEEFLEENMEPGVKEVPEGNSKDLDACSQLSAGPHKSHLHPGSSGRERCSPPPGGASAGGAQGPGGGPTPDGPIPVLLQIQPVPVKQESGTGPASPGQAPENVKVAQLLVNIQGQTFALVPQVVPSSNLNLPSKFVRIAPVPIAAKPVGSGPLGPGPAGLLMGQKFPKNPAAELIKMHKCTFPGCSKMYTKSSHLKAHLRRHTGEKPFACTWPGCGWRFSRSDELSRHRRSHSGVKPYQCPVCEKKFARSDHLSKHIKVHRFPRSSRSVRSVN

SEQ ID NO: 23 - protein, 211 aa, Homo sapiens [sequence omitted]

SEQ ID NO: 24 - protein, 389 aa, Homo sapiens
MYGRPQAEMEQEAGELSRWQAAHQAAQDNENSAPILNMSSSSGSSGVHTSWNQGLPTIQHFPHSAEMLGSPLVSVEAPGQNVNEGGPQFSMPLPERGMSYCPQATLTPSRMIYCQRMSPPQQEMTIFSGPQLMPVGEPNIPRVARPFGGNLRMPPSGLPVSASTGIPIMSHTGNPPVPYPGLSTVPSDETLLGPTVPSTEAQAVLPSMAQMLPPQDAHDLGMPPAESQSLLVLGSQDSLVSQPDSQEGPFLPEQPGPAPQTVEKNSRPQEGTGRRGSSEARPYCCNYENCGKAYTKRSHLVSHQRKHTGERPYSCNWESCSWSFFRSDELRRHMRVHTRYRPYKCDQCSREFMRSDHLKQHQKTHRPGPSDPQANNNNGEQDSPPAAGP

SEQ ID NO: 25 - DNA, 20 bp, Artificial Sequence
  Ce-klf-1 specific primer
gccacgtcatcacgggaacc

SEQ ID NO: 26 - DNA, 20 bp, Artificial Sequence
  Ce-klf-1 specific primer
ctccgagagctgtcgtcggt

SEQ ID NO: 27 - DNA, 20 bp, Artificial Sequence
  ama-1 specific primer
gcattgtctcacgcgttcag

SEQ ID NO: 28 - DNA, 20 bp, Artificial Sequence
  ama-1 specific primer
ttcttccttctccgctgctc

SEQ ID NO: 29 - DNA, 18 bp, Artificial Sequence
  C. elegans klf-3 primer
ccactacatcaagcgagc

SEQ ID NO: 30 - DNA, 18 bp, Artificial Sequence
  C. elegans klf-3 primer
gcgcttcatgtgaagact

SEQ ID NO: 31 - DNA, 18 bp, Artificial Sequence
  acs-1 primer
tatccaccaccaccagtg

SEQ ID NO: 32 - DNA, 18 bp, Artificial Sequence
  acs-1 primer
atacatagggtagggggg

SEQ ID NO: 33 - DNA, 21 bp, Artificial Sequence
  acs-2 primer
atgtcgctgatgctcatgtcg

SEQ ID NO: 34 - DNA, 20 bp, Artificial Sequence
  acs-1 primer
cagttccgagacccaacagc

SEQ ID NO: 35 - DNA, 18 bp, Artificial Sequence
  acs-3 primer
aaatggcttccaaccggc

SEQ ID NO: 36 - DNA, 19 bp, Artificial Sequence
  acs-3 primer
tttccgtccaacgccttca

SEQ ID NO: 37 - DNA, 19 bp, Artificial Sequence
  acs-11 primer
aactgttggcccggctgta

SEQ ID NO: 38 - DNA, 21 bp, Artificial Sequence
  acs-11 primer
ctccgacgggactacaattgc

SEQ ID NO: 39 - DNA, 21 bp, Artificial Sequence
  cpt-5 primer
tcccgcaggaagttattgaaa

SEQ ID NO: 40 - DNA, 20 bp, Artificial Sequence
  cpt-5 primer
gcttgatttcctccgaatcg

SEQ ID NO: 41 - DNA, 21 bp, Artificial Sequence
  dhs-25 primer
ctaaatccaccggtaacttcc

SEQ ID NO: 42 - DNA, 17 bp, Artificial Sequence
  dhs-25 primer
caaggccggagtcatcg

SEQ ID NO: 43 - DNA, 19 bp, Artificial Sequence
  ech-1 primer
aaccaagaggcggcaaagc

SEQ ID NO: 44 - DNA, 20 bp, Artificial Sequence
  ech-1 primer
gttggcatggctcaaattgg

SEQ ID NO: 45 - DNA, 20 bp, Artificial Sequence
  ech-8 primer
tcaattccttgaagccatcc

SEQ ID NO: 46 - DNA, 19 bp, Artificial Sequence
  ech-8 primer
gaacgatcaggatgccgtc

SEQ ID NO: 47 - DNA, 20 bp, Artificial Sequence
  ech-9 primer
gagcaatcctctcaacggtg

SEQ ID NO: 48 - DNA, 21 bp, Artificial Sequence
  ech-9 primer
ccggtgtattgaagaaggtgt

SEQ ID NO: 49 - DNA, 20 bp, Artificial Sequence
  elo-2 primer
gattctgttcctggttgcgc

SEQ ID NO: 50 - DNA, 21 bp, Artificial Sequence
  elo-2 primer
gacatgcccgtaagagtggaa

SEQ ID NO: 51 - DNA, 21 bp, Artificial Sequence
  elo-6 primer
tcaaggttccagcatggattg

SEQ ID NO: 52 - DNA, 20 bp, Artificial Sequence
  elo-6 primer
tcttgccacctcccttgatg

SEQ ID NO: 53 - DNA, 22 bp, Artificial Sequence
  fasn-1 primer
tctcatccaatctctcccctca

SEQ ID NO: 54 - DNA, 20 bp, Artificial Sequence
  fasn-1 primer
ttgaaatcaagggtgggcag

SEQ ID NO: 55 - DNA, 19 bp, Artificial Sequence
  fat-1 primer
acggacacgttgcccatca

SEQ ID NO: 56 - DNA, 20 bp, Artificial Sequence
  fat-1 primer
gcctttgccttctcctcgag

SEQ ID NO: 57 - DNA, 20 bp, Artificial Sequence
  fat-2 primer
attaccaacggtcacgtcgc

SEQ ID NO: 58 - DNA, 20 bp, Artificial Sequence
  fat-2 primer
gcctttgcagcctcaactcc

SEQ ID NO: 59 - DNA, 19 bp, Artificial Sequence
  fat-3 primer
accaacatggccacttcgg

SEQ ID NO: 60 - DNA, 20 bp, Artificial Sequence
  fat-3 primer
cattcagattgcaacgtggc

SEQ ID NO: 61 - DNA, 21 bp, Artificial Sequence
  fat-4 primer
tggaggtttcctgctctctca

SEQ ID NO: 62 - DNA, 21 bp, Artificial Sequence
  fat-4 primer
tggtaaaccatttgctgctgc

SEQ ID NO: 63 - DNA, 20 bp, Artificial Sequence
  fat-5 primer
acgctacatggtgcatcaac

SEQ ID NO: 64 - DNA, 19 bp, Artificial Sequence
  fat-5 primer
agccgaacttcttgcactg

SEQ ID NO: 65 - DNA, 21 bp, Artificial Sequence
  fat-6 primer
ctaccagctcatcttcgaggc

SEQ ID NO: 66 - DNA, 22 bp, Artificial Sequence
  fat-6 primer
gatcacgagcccattcgatgac

SEQ ID NO: 67 - DNA, 19 bp, Artificial Sequence
  fat-7 primer
cgatacttctgtttccgcc

SEQ ID NO: 68 - DNA, 20 bp, Artificial Sequence
  fat-7 primer
ttcttgattcttcacttccg

SEQ ID NO: 69 - DNA, 19 bp, Artificial Sequence
  pod-2 primer
tcggtcgagtttgcggatg

SEQ ID NO: 70 - DNA, 20 bp, Artificial Sequence
  pod-2 primer
tcgtccattgagctgttcgg

SEQ ID NO: 71 - DNA, 20 bp, Artificial Sequence
  B0272.3 primer
ccgtctcttggtgccttaca

SEQ ID NO: 72 - DNA, 20 bp, Artificial Sequence
  B0272.3 primer
tcggctagcaatcatcattc

SEQ ID NO: 73 - DNA, 19 bp, Artificial Sequence
  B0303.3 primer
atcggacatccattcggag

SEQ ID NO: 74 - DNA, 18 bp, Artificial Sequence
  B0303.3 primer
aaggcacgacaaccgcta

SEQ ID NO: 75 - DNA, 20 bp, Artificial Sequence
  C17C3.4 primer
ttctcagcagctggtcattg

SEQ ID NO: 76 - DNA, 19 bp, Artificial Sequence
  C17C3.4 primer
gctccagaagtggcttgca

SEQ ID NO: 77 - DNA, 20 bp, Artificial Sequence
  C48B4.1 primer
aagttgtttatcgcccgtgg

SEQ ID NO: 78 - DNA, 20 bp, Artificial Sequence
  C48B4.1 primer
atcacggcgaccgagtactg

SEQ ID NO: 79 - DNA, 20 bp, Artificial Sequence
  F08A8.1 primer
cgtagacatgaccatcacgg

SEQ ID NO: 80 - DNA, 20 bp, Artificial Sequence
  F08A8.1 primer
caagtcatccgtggagttga

SEQ ID NO: 81 - DNA, 19 bp, Artificial Sequence
  F08A8.2 primer
agcctgccttctagccatg

SEQ ID NO: 82 - DNA, 19 bp, Artificial Sequence
  F08A8.2 primer
ggattgctattggcggatg

SEQ ID NO: 83 - DNA, 18 bp, Artificial Sequence
  F38H4.8 primer
agcgcgattgcactacgt

SEQ ID NO: 84 - DNA, 20 bp, Artificial Sequence
  F38H4.8 primer
tctgaagctcaaggattgcc

SEQ ID NO: 85 - DNA, 21 bp, Artificial Sequence
  F44C4.5 primer
ttgcaagtgatctccatccac

SEQ ID NO: 86 - DNA, 20 bp, Artificial Sequence
  F44C4.5 primer
acccgacttacaaacgcaac

SEQ ID NO: 87 - DNA, 17 bp, Artificial Sequence
  F53A2.7 primer
tacttgacgttgcggcg

SEQ ID NO: 88 - DNA, 19 bp, Artificial Sequence
  F53A2.7 primer
ctgctgtcggttgtgatcc

SEQ ID NO: 89 - DNA, 21 bp, Artificial Sequence
  F54C8.1 primer
acgagtagaatccgtcaccag

SEQ ID NO: 90 - DNA, 20 bp, Artificial Sequence
  F54C8.1 primer
aggagatgcatcaatgaccg

SEQ ID NO: 91 - DNA, 19 bp, Artificial Sequence
  F59F4.1 primer
cttcgagatggcacgttcg

SEQ ID NO: 92 - DNA, 19 bp, Artificial Sequence
  F59F4.1 primer
caaccgcatttggacgcat

SEQ ID NO: 93 - DNA, 20 bp, Artificial Sequence
  K05F1.3 primer
aagttctggaacagtgtgcg

SEQ ID NO: 94 - DNA, 19 bp, Artificial Sequence
  K05F1.3 primer
tcatgattgcggatatggc

SEQ ID NO: 95 - DNA, 18 bp, Artificial Sequence
  R06F6.9 primer
tgctgcaatcgcttggtg

SEQ ID NO: 96 - DNA, 21 bp, Artificial Sequence
  R06F6.9 primer
gcttctgagatgctgttggtc

SEQ ID NO: 97 - DNA, 20 bp, Artificial Sequence
  R07C3.4 primer
tgagttggagttgtcaagcc

SEQ ID NO: 98 - DNA, 20 bp, Artificial Sequence
  R07C3.4 primer
ctcgtagcagtcgtcgttcc

SEQ ID NO: 99 - DNA, 19 bp, Artificial Sequence
  R07H5.2 primer
cgattgagccaaccaactc

SEQ ID NO: 100 - DNA, 19 bp, Artificial Sequence
  R07H5.2 primer
tcggatcgagaaggtgacc

SEQ ID NO: 101 - DNA, 19 bp, Artificial Sequence
  R09E10.3 primer
aagcaactggcgtcaagtg

SEQ ID NO: 102 - DNA, 20 bp, Artificial Sequence
  R09E10.3 primer
ttacgttcatggcgatatgg

SEQ ID NO: 103 - DNA, 18 bp, Artificial Sequence
  T05G5.6 primer
ctcttctcggcaaaagcg

SEQ ID NO: 104 - DNA, 19 bp, Artificial Sequence
  T05G5.6 primer
ccatggaggtgtgccttac

SEQ ID NO: 105 - DNA, 20 bp, Artificial Sequence
  T08B2.7 primer
tcgcgaagatcaagaagaga

SEQ ID NO: 106 - DNA, 20 bp, Artificial Sequence
  T08B2.7 primer
caatgaggcgcttctatgtc

SEQ ID NO: 107 - DNA, 28 bp, Artificial Sequence
  Ce-klf-3 primer
tcaagcttatgctgaaaatggaacaaag

SEQ ID NO: 108 - DNA, 26 bp, Artificial Sequence
  Ce-klf-3 primer
caggatccattgtgctatggcgcttc

SEQ ID NO: 109 - protein, 309 aa, Caenorhabditis elegans
MTSPNIFQDWADVYEFIERDSSRKNVPAIEAIERRLAFSPLITPNPGAKQFAPIHVPGREPPRMLLPPTPHFQAPFSPHPPPVQQVPSYSPPHAPPSYETYPEVYYPPHIICNPYDVPTTSDRNPPYYTEVTTVSAVTLHSMTPPTHKIETPPSSPENSFGPLASQLPAIKMEIPMHPLPHNGELDSTRSSPSSTTSSERSPLQRKSRIESNKRNPTDKKFVVHACTYPGCFKKYSKSSHLKAHERTHSGEKPFVCKWQNCSWKFARSDELTRHMRKHTGDKPFRCSLCDRNFARSDHLSLHMKRHSTI

SEQ ID NO: 110 - protein, 315 aa, Caenorhabditis elegans
MLKMEQSAPPRYEEDWADVYEFIERDSSRKNVPAIEAIERRLAFSPLITPNPGAKQFAPIHVPGREPPRMLLPPTPHFQAPFSPHPPPVQQVPSYSPPHAPPSYETYPEVYYPPHIICNPYDVPTTSDRNPPYYTEVTTVSAVTLHSMTPPTHKIETPPSSPENSFGPLASQLPAIKMEIPMHPLPHNGELDSTRSSPSSTTSSERSPLQRKSRIESNKRNPTDKKFVVHACTYPGCFKKYSKSSHLKAHERTHSGEKPFVCKWQNCSWKFARSDELTRHMRKHTGDKPFRCSLCDRNFARSDHLSLHMKRHSTI